(19) United States
(12) Patent Application Publication
(10) Pub. No.: US 2022/0226243 A1
(43) Pub. Date: Jul. 21, 2022

(54) METHODS OF USING LIPID NANOPARTICLES FOR DELIVERING MODIFIED RNA ENCODING A VEGF-A POLYPEPTIDE AND PHARMACEUTICAL COMPOSITIONS COMPRISING THE SAME

(71) Applicant: AstraZeneca AB, Södertälje (SE)

(72) Inventors: Kenny Mikael Hansson, MöIndal (SE); Maria Wågberg, MöIndal (SE); Nils Bergenhem, Waltham, MA (US)

(73) Assignee: AstraZeneca AB, Södertälje (SE)

(21) Appl. No.: 17/609,258

(22) PCT Filed: May 8, 2020
(86) PCT No.: PCT/US2020/032241

§ 371 Date: Nov. 5, 2021

Related U.S. Application Data
(60) Provisional application No. 62/845,184, filed on May 8, 2019.

(51) Int. Cl. A61K 9/127; A61K 38/18; A61K 48/00; A61P 17/02

(52) U.S. Cl. CPC A61K 9/1272; A61P 17/02; A61K 48/0075; A61K 38/1866

(57) ABSTRACT

The disclosure relates to nanoparticles comprising a lipid component and a modified RNA encoding a VEGF-A polypeptide. Aspects of the disclosure further relate to uses of nanoparticles comprising a lipid component and a modified RNA encoding a VEGF-A polypeptide, for improving wound healing in a subject. Some aspects of the disclosure relate to the topical administration of nanoparticles comprising a lipid component and a modified RNA.

### 1. SEQUENCE LISTING

The instant application contains a Sequence Listing which has been submitted electronically in ASCII format and is hereby incorporated by reference in its entirety. Said ASCII copy, created on May 8, 2019, is named 09963_6016-00000_SL.txt and is 11,396 bytes in size.

### 2. FIELD

The disclosure relates to nanoparticles comprising a lipid component and a modified RNA encoding a VEGF-A polypeptide. Aspects of the disclosure further relate to uses of nanoparticles comprising a lipid component and a modified RNA encoding a VEGF-A polypeptide for improving wound healing in a subject.

### 3. BACKGROUND

Vascular endothelial growth factor A (VEGF-A) pathways play a central role in the wound healing process, including revascularization of damaged tissues, improving vascular permeability, and formation of new blood vessels (angiogenesis). It remains challenging to deliver agents to augment VEGF-A pathways for potential therapeutic effects such as improving wound healing in a subject.

A diverse number of methods has been attempted to allow clinically tractable approaches to increase VEGF-A proteins in target tissues. However, each of the approaches has significant drawbacks. For instance, systemic VEGF-A protein delivery can result in significant hypotension and VEGF-A is rapidly degraded. Viral encapsulated and naked VEGF-A DNA plasmids have limited temporal control of protein expression and the efficiency of in vivo expression can be highly variable and non-dose dependent. As a result, these limitations have restricted the applicability of augmenting VEGF-A levels as a therapeutic agent.

Another recent development is to deliver therapeutic RNAs encoding VEGF-A proteins. However, delivery of natural RNAs to cells can be challenging due to the relative instability and low cell permeability of such RNA molecules. Also, natural RNAs can trigger immune activation (See, e.g., Kaczmarek et al., “Advances in the delivery of RNA therapeutics: from concept to clinical reality,” Genome Med., 2017, 9: 60), which limit their uses for delivering VEGF-A proteins to target tissues.

Accordingly, there remains a need for compositions that allow for effective and safe delivery of RNAs encoding VEGF-A proteins. In addition, there remains a need for alternative methods to augment VEGF-A pathways for potential therapeutic effects such as improving wound healing in a subject.

### 4. SUMMARY

The disclosure relates to nanoparticles comprising a lipid component and a modified RNA encoding a VEGF-A polypeptide. Aspects of the disclosure further relate to uses of nanoparticles comprising a lipid component and a modified RNA encoding a VEGF-A polypeptide, for improving wound healing in a subject.

Certain embodiments of the present disclosure are summarized in the following paragraphs. This list is only exemplary and not exhaustive of all of the embodiments provided by this disclosure. In some aspects, the present disclosure relates to the following embodiments:

1. A nanoparticle comprising

(i) a lipid component comprising dilinoleylmethyl-4-dimethylaminobutyrate (DLin-MC3-DMA), and

(ii) a modified RNA comprising any one of SEQ ID NOs: 1 and 3-5, encoding a VEGF-A polypeptide of SEQ ID NO: 2.

2. The nanoparticle according to embodiment 1, wherein the lipid component further comprises a phospholipid, a structural lipid, and/or a PEG lipid.

3. The nanoparticle according to embodiment 1 or 2, wherein the lipid component further comprises a phospholipid, a structural lipid, and a PEG lipid.

4. The nanoparticle according to embodiment 2 or 3, wherein the phospholipid is selected from the group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof;

the structural lipid is selected from the group consisting of cholesterol, fecosterol, sitosterol, ergosterol, campesterol, stigmasterol, brassicasterol, tomatidine, ursolic acid, alpha-tocopherol, and mixtures thereof; and/or

the PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, DMG-PEG (1,2-dimyristoyl-rac-glycero-3-methoxypolyethylene glycol), DMG-PEG2000 (1,2-dimyristoyl-rac-glycero-3-methoxypolyethylene glycol-2000), and mixtures thereof.

5. The nanoparticle according to any one of embodiments 1-4, wherein the lipid component further comprises a phospholipid that is DSPC, a structural lipid that is cholesterol, and/or a PEG lipid that is DMG-PEG.

6. The nanoparticle according to any one of embodiments 1-5, wherein the ratio of ionizable nitrogen atoms in the lipid to the number of phosphate groups in the RNA (N:P ratio) is from about 2:1 to about 30:1.

7. The nanoparticle of embodiment 6, wherein the N:P ratio is about 3:1.

8. The nanoparticle according to any one of embodiments 1-7, wherein the wt/wt ratio of the lipid component to the modified RNA is from about 5:1 to about 100:1.

9. The nanoparticle of embodiment 8, wherein the wt/wt ratio of the lipid component to the modified RNA is about 10:1.

10. The nanoparticle according to any one of embodiments 1-9 wherein the nanoparticle has a mean diameter from about 50 nm to about 100 nm.

11. The nanoparticle according to any one of embodiments 1-9 wherein the nanoparticle has a mean diameter from about 70 nm to about 90 nm.

12. The nanoparticle according to embodiment 11, wherein the nanoparticle has a mean diameter of about 70 nm to about 85 nm.

13. A pharmaceutical composition comprising

(a) at least one nanoparticle comprising (i) a lipid component comprising dilinoleylmethyl-4-dimethylaminobutyrate (DLin-MC3-DMA), and (ii) a modified RNA comprising any one of SEQ ID NOs: 1 and 3-5, encoding a VEGF-A polypeptide of SEQ ID NO: 2; and

(b) a pharmaceutically acceptable excipient.

14. The pharmaceutical composition of embodiment 13, wherein the lipid component further comprises a phospholipid, a structural lipid, and/or a PEG lipid.

15. The pharmaceutical composition of according to embodiments 13 or 14, wherein the lipid component further comprises a phospholipid, a structural lipid, and a PEG lipid.

16. The pharmaceutical composition according to embodiments 14 or 15, wherein the phospholipid is selected from the group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof;

the structural lipid is selected from the group consisting of cholesterol, fecosterol, sitosterol, ergosterol, campesterol, stigmasterol, brassicasterol, tomatidine, ursolic acid, alpha-tocopherol, and mixtures thereof; and/or

the PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, DMG-PEG (1,2-dimyristoyl-rac-glycero-3-methoxypolyethylene glycol), DMG-PEG2000 (1,2-dimyristoyl-rac-glycero-3-methoxypolyethylene glycol-2000), and mixtures thereof.

17. The pharmaceutical composition according to any one of embodiments 13-16, wherein the lipid component further comprises a phospholipid that is DSPC, a structural lipid that is cholesterol, and/or a PEG lipid that is DMG-PEG.

18. The pharmaceutical composition according to any one of embodiments 13-17, wherein the ratio of ionizable nitrogen atoms in the lipid to the number of phosphate groups in the RNA (N:P ratio) is from about 2:1 to about 30:1.

19. The pharmaceutical composition of embodiment 18, wherein the N:P ratio is about 3:1.

20. The pharmaceutical composition according to any one of embodiments 13-19, wherein the wt/wt ratio of the lipid component to the modified RNA is from about 5:1 to about 100:1.

21. The pharmaceutical composition of embodiment 20, wherein the wt/wt ratio of the lipid component to the modified RNA is about 10:1.

22. The pharmaceutical composition according to any one of embodiments 13-21, wherein the nanoparticle has a mean diameter from about 50 nm to about 100 nm.

23. The pharmaceutical composition according to any one of embodiments 13-21, wherein the nanoparticle has a mean diameter from about 70 nm to about 90 nm.

24. The pharmaceutical composition of embodiment 23, wherein the nanoparticle has a mean diameter of about 70 nm to about 85 nm.

25. The pharmaceutical composition of any one of embodiments 13-24, wherein the pharmaceutically acceptable excipient is chosen from a solvent, dispersion media, diluent, dispersion, suspension aid, surface active agent, isotonic agent, thickening or emulsifying agent, preservative, polymer, peptide, protein, cell, hyaluronidase, and mixtures thereof.

26. A method for promoting and/or improving wound healing, comprising administering to a subject in need thereof an effective amount of the nanoparticle according to any one of embodiments 1-12 or the pharmaceutical composition according to any one of embodiments 13-25.

27. The method of embodiment 26, wherein the administration results in production of a VEGF-A polypeptide of SEQ ID NO: 2 in plasma or tissue of the subject.

28. The method of embodiment 27, wherein the VEGF-A polypeptide is detected in the plasma and/or tissue within 5 or 6 hours after administration of the nanoparticle or pharmaceutical composition to the subject.

29. The method of embodiments 27 or 28, wherein the administration results in production of more than about 1 pg/mg of the VEGF-A polypeptide in the subject.

30. The method according to any one of embodiments 26-29, wherein the nanoparticle or the pharmaceutical composition is administered intradermally.

31. The method according to any one of embodiments 26-29, wherein the nanoparticle or the pharmaceutical composition is administered topically to a wound.

32. The method according to any one of embodiments 26-31, wherein the nanoparticle is administered at a dosage level sufficient to deliver from about 0.01 mg/kg to about 10 mg/kg of modified RNA per subject body weight.

33. The method according to any one of embodiments 26-32, wherein the administration increases production of a VEGF-A polypeptide of SEQ ID NO: 2 by a factor of about 1 to about 100, as compared to administration of the modified RNA in a citrate saline buffer to the subject.

34. The method according to any one of embodiments 26-33, wherein the subject suffers from diabetes.

35. The method according to any one of embodiments 26-34, wherein the wound is a surgical wound, a burn, an abrasive wound, a skin biopsy site, a chronic wound, an injury (e.g., a traumatic injury wound), a graft wound, a diabetic wound, a diabetic ulcer (e.g., diabetic foot ulcer), a pressure ulcer, bed sore, and combinations thereof.

36. A method for inducing neovascularization comprising administering to a subject in need thereof an effective amount of the nanoparticle according to any one of embodiments 1-12 or the pharmaceutical composition according to any one of embodiments 13-25.

37. A method for inducing angiogenesis comprising administering to a subject in need thereof an effective amount of the nanoparticle according to any one of embodiments 1-12 or the pharmaceutical composition according to any one of embodiments 13-25.

38. A method for increasing capillary and/or arteriole density comprising administering to a subject in need thereof an effective amount of the nanoparticle according to any one of embodiments 1-12 or the pharmaceutical composition according to any one of embodiments 13-25.

39. A method for promoting and/or improving wound healing, comprising topically administering to a wound in a subject in need thereof an effective amount of a nanoparticle or pharmaceutical composition thereof comprising

(i) a lipid component, and

(ii) a modified RNA comprising any one of SEQ ID NOs: 1 and 3-5, encoding a VEGF-A polypeptide of SEQ ID NO: 2.

40. The method of claim 39, wherein the lipid component comprises a compound having the structure

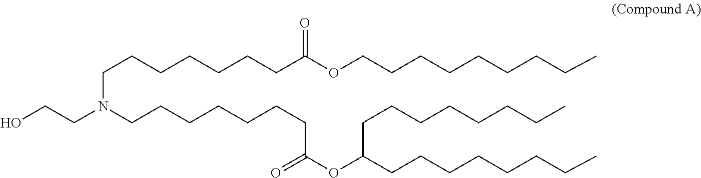 [SMILES: CCCCCCCCCOC(=O)CCCCCCCN(CCO)CCCCCCCC(=O)OC(CCCCCCCC)CCCCCCCC]

41. The method of claim 39, wherein the lipid component comprises dilinoleylmethyl-4-dimethylaminobutyrate.

42. A method for promoting and/or improving wound healing, comprising topically administering to a wound in a subject in need thereof an effective amount of a nanoparticle or a pharmaceutical composition thereof comprising

(i) a lipid component comprising a compound having the structure

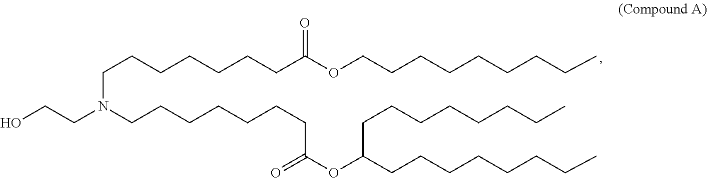 [SMILES: CCCCCCCCCOC(=O)CCCCCCCN(CCO)CCCCCCCC(=O)OC(CCCCCCCC)CCCCCCCC]

and

(ii) a modified RNA comprising any one of SEQ ID NOs: 1 and 3-5, encoding a VEGF-A polypeptide of SEQ ID NO: 2.

43. A method for promoting and/or improving wound healing, comprising topically administering to a wound in a subject in need thereof an effective amount of a nanoparticle or a pharmaceutical composition thereof comprising

(i) a lipid component comprising dilinoleylmethyl-4-dimethylaminobutyrate, and

(ii) modified RNA comprising any one of SEQ ID NOs: 1 and 3-5, encoding a VEGF-A polypeptide of SEQ ID NO: 2.

44. The method according to any one of embodiments 39-43, wherein the lipid component further comprises a phospholipid, a structural lipid, and a PEG lipid.

45. The method according to any one of embodiments 39-44, wherein the lipid component further comprises a phospholipid that is DSPC, a structural lipid that is cholesterol, and/or a PEG lipid that is DMG-PEG.

46. The method according to any one of embodiments 39-45, wherein the ratio of ionizable nitrogen atoms in the lipid to the number of phosphate groups in the RNA (N:P ratio) is about 3:1.

47. The method according to any one of embodiments 39-46, wherein the wt/wt ratio of the lipid component to the modified RNA is about 10:1.

48. The method according to any one of embodiments 39-47, wherein the pharmaceutical composition comprises a pharmaceutically acceptable excipient chosen from a solvent, dispersion media, diluent, dispersion, suspension aid, surface active agent, isotonic agent, thickening or emulsifying agent, preservative, polymer, peptide, protein, cell, hyaluronidase, and mixtures thereof.

49. The method according to any one of embodiments 39-48, wherein the administration results in production of a VEGF-A polypeptide of SEQ ID NO: 2 in plasma or tissue of the subject.

50. The method according to any one of embodiments claim 39-49, wherein the administration increases production of a VEGF-A polypeptide of SEQ ID NO:2, as compared to administration of the modified RNA in a citrate saline buffer to the subject.

51. The method according to any one of embodiments 39-50 wherein the subject suffers from diabetes.

52. The method according to any one of embodiments 39-51 wherein the wound is a surgical wound, a burn, an abrasive wound, a skin biopsy site, a chronic wound, an injury (e.g., a traumatic injury wound), a graft wound, a diabetic wound, a diabetic ulcer (e.g., diabetic foot ulcer), a pressure ulcer, bed sore, and combinations thereof.

53. The nanoparticle according to any one of embodiments 1-12 or the pharmaceutical composition according to any one of embodiments 13-25 for use in a method for promoting and/or improving wound healing, comprising administering to a subject in need thereof an effective amount of the nanoparticle or pharmaceutical composition.

54. The nanoparticle or pharmaceutical composition for use of embodiment 53, wherein the administration results in production of a VEGF-A polypeptide of SEQ ID NO: 2 in plasma or tissue of the subject.

55. The nanoparticle or pharmaceutical composition for use of embodiment 54, wherein the VEGF-A polypeptide is detected in the plasma and/or tissue within 5 or 6 hours after administration of the nanoparticle or pharmaceutical composition to the subject.

56. The nanoparticle or pharmaceutical composition for use according to any one of embodiments 54 or 55, wherein the administration results in production of more than about 1 pg/mg of the VEGF-A polypeptide in the subject.

57. The nanoparticle or pharmaceutical composition for use according to any one of embodiments 53-56, wherein the nanoparticle or the pharmaceutical composition is administered intradermally.

58. The nanoparticle or pharmaceutical composition for use according to any one of embodiments 53-56, wherein the nanoparticle or the pharmaceutical composition is administered topically to a wound.

59. The nanoparticle or pharmaceutical composition for use according to any one of embodiments 53-58, wherein the nanoparticle is administered at a dosage level sufficient to deliver from about 0.01 mg/kg to about 10 mg/kg of modified RNA per subject body weight.

60. The nanoparticle or pharmaceutical composition for use according to any one of embodiments 53-59, wherein the administration increases production of a VEGF-A polypeptide of SEQ ID NO: 2 by a factor of about 1 to about 100, as compared to administration of the modified RNA in a citrate saline buffer to the subject.

61. The nanoparticle or pharmaceutical composition for use according to any one of embodiments 53-60, wherein the subject suffers from diabetes.

62. The nanoparticle or pharmaceutical composition for use according to any one of embodiments 53-61, wherein the wound is a surgical wound, a burn, an abrasive wound, a skin biopsy site, a chronic wound, an injury (e.g., a traumatic injury wound), a graft wound, a diabetic wound, a diabetic ulcer (e.g., diabetic foot ulcer), a pressure ulcer, bed sore, and combinations thereof.

63. The nanoparticle according to any one of embodiments 1-12 or the pharmaceutical composition according to any one of embodiments 13-25 for use in a method for inducing neovascularization.

64. The nanoparticle according to any one of embodiments 1-12 or the pharmaceutical composition according to any one of embodiments 13-25 for use in a method for inducing angiogenesis.

65. The nanoparticle according to any one of embodiments 1-12 or the pharmaceutical composition according to any one of embodiments 13-25 for use in a method for increasing capillary and/or arteriole density.

66. A nanoparticle or pharmaceutical composition thereof for use in a method for promoting and/or improving wound healing, comprising topically administering to a wound in a subject in need thereof an effective amount of the nanoparticle or pharmaceutical composition, wherein the nanoparticle or pharmaceutical composition thereof comprises

(i) a lipid component, and

(ii) a modified RNA comprising any one of SEQ ID NOs: 1 and 3-5, encoding a VEGF-A polypeptide of SEQ ID NO: 2.

67. The nanoparticle or pharmaceutical composition for use of claim 66, wherein the lipid component comprises a compound having the structure

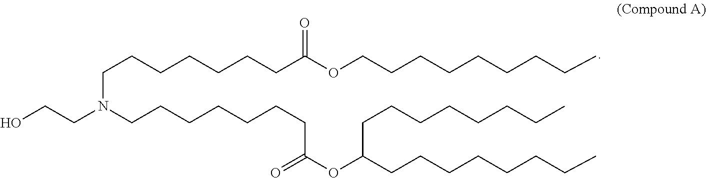 [SMILES: CCCCCCCCCOC(=O)CCCCCCCN(CCO)CCCCCCCC(=O)OC(CCCCCCCC)CCCCCCCC]

68. The nanoparticle or pharmaceutical composition for use of claim 66, wherein the lipid component comprises dilinoleylmethyl-4-dimethylaminobutyrate.

69. A nanoparticle or a pharmaceutical composition thereof for use in a method for promoting and/or improving wound healing, comprising topically administering to a wound in a subject in need thereof an effective amount of the nanoparticle or pharmaceutical composition, wherein the nanoparticle or pharmaceutical composition thereof comprises

(i) a lipid component comprising a compound having the structure

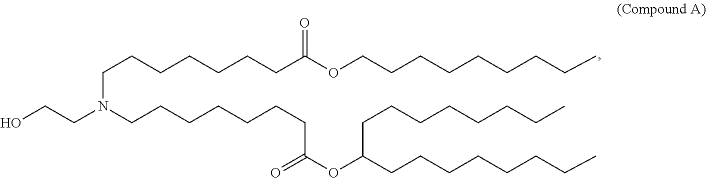 [SMILES: CCCCCCCCCOC(=O)CCCCCCCN(CCO)CCCCCCCC(=O)OC(CCCCCCCC)CCCCCCCC]

and

(ii) a modified RNA comprising any one of SEQ ID NOs: 1 and 3-5, encoding a VEGF-A polypeptide of SEQ ID NO: 2.

70. A nanoparticle or a pharmaceutical composition thereof for use in a method for promoting and/or improving wound healing, comprising topically administering to a wound in a subject in need thereof an effective amount of the nanoparticle or pharmaceutical composition, wherein the nanoparticle or pharmaceutical composition thereof comprises

(i) a lipid component comprising dilinoleylmethyl-4-dimethylaminobutyrate, and

(ii) modified RNA comprising any one of SEQ ID NOs: 1 and 3-5, encoding a VEGF-A polypeptide of SEQ ID NO: 2.

71. The nanoparticle or pharmaceutical composition for use according to any one of embodiments 66-70, wherein the lipid component further comprises a phospholipid, a structural lipid, and a PEG lipid.

72. The nanoparticle or pharmaceutical composition for use according to any one of embodiments 66-71, wherein the lipid component further comprises a phospholipid that is DSPC, a structural lipid that is cholesterol, and/or a PEG lipid that is DMG-PEG.

73. The nanoparticle or pharmaceutical composition for use according to any one of embodiments 66-72, wherein the ratio of ionizable nitrogen atoms in the lipid to the number of phosphate groups in the RNA (N:P ratio) is about 3:1.

74. The nanoparticle or pharmaceutical composition for use according to any one of embodiments 66-73, wherein the wt/wt ratio of the lipid component to the modified RNA is about 10:1.

75. The pharmaceutical composition for use according to any one of embodiments 66-74, wherein the pharmaceutical composition comprises a pharmaceutically acceptable excipient chosen from a solvent, dispersion media, diluent, dispersion, suspension aid, surface active agent, isotonic agent, thickening or emulsifying agent, preservative, polymer, peptide, protein, cell, hyaluronidase, and mixtures thereof.

76. The nanoparticle or pharmaceutical composition for use according to any one of embodiments 66-75, wherein the administration results in production of a VEGF-A polypeptide of SEQ ID NO: 2 in plasma or tissue of the subject.

77. The nanoparticle or pharmaceutical composition for use according to any one of embodiments claim 66-76, wherein the administration increases production of a VEGF-A polypeptide of SEQ ID NO:2, as compared to administration of the modified RNA in a citrate saline buffer to the subject.

78. The nanoparticle or pharmaceutical composition for use according to any one of embodiments 66-77 wherein the subject suffers from diabetes.

79. The nanoparticle or pharmaceutical composition for use according to any one of embodiments 66-78 wherein the wound is a surgical wound, a burn, an abrasive wound, a skin biopsy site, a chronic wound, an injury (e.g., a traumatic injury wound), a graft wound, a diabetic wound, a diabetic ulcer (e.g., diabetic foot ulcer), a pressure ulcer, bed sore, and combinations thereof.

### 5. DESCRIPTION OF DRAWINGS

Those of skill in the art will understand that the drawings, described below, are for illustrative purposes only. The drawings are not intended to limit the scope of the present teachings in any way.

FIG. 1: FIG. 1 shows the lipid compound (Compound A) used in the Examples.

FIGS. 2A and 2B: A diagram of the structure (FIG. 2A) of a modified VEGF-A RNA construct and the sequence (SEQ ID NO: 1, FIG. 2B) of a representative VEGF-A modified RNA.

FIG. 3 shows the lipid compound dilinoleylmethyl-4-dimethylaminobutyrate (DLin-MC3-DMA).

FIG. 4: Study timeline for the assessment of wound healing following intradermal injection of a modified VEGF-A RNA in mouse.

FIG. 5: Effect of intradermal administration (injection) of a modified VEGF-A RNA formulated with MC3 (mRNA VEGF 3 μg MC3), a non-translatable VEGF-A RNA formulated with MC3 (mRNAVEGF NT (3 μg) MC3), and a saline/citrate composition on wound healing.

FIG. 6: Study timeline for the assessment of wound healing following topical administration of a modified VEGF-A RNA in mouse.

FIG. 7: Effect of topical administration of a modified VEGF-A RNA formulated with MC3 (mRNA VEGF (3 μg) MC3), and a saline/citrate composition on wound healing.

FIG. 8A: Human VEGF-A (hVEGF-A) protein expression in pig tissue 5-6 hours after topical administration of a modified VEGF-A RNA formulated with Compound A, topical administration of modified VEGF-A RNA formulated in saline/citrate, topical administration of a modified VEGF-A RNA formulated with MC3, and intradermal (single inj) administration of modified VEGF-A formulated with MC3.

FIG. 8B: Picture of a wound on pig skin, with drawn circles indicating sites of topical administration.

### 6. DETAILED DESCRIPTION

All references referred to in this disclosure are incorporated herein by reference in their entireties.

Many modifications and other embodiments of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. Although specific terms are employed herein, they are used in a generic and descriptive sense only and not for purposes of limitation.

Units, prefixes and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range. The recitation of ranges of values herein is merely intended to serve as a shorthand method of referring individually to each separate value falling within the range. Unless otherwise indicated herein, each individual value is incorporated into the specification as if it were individually recited herein. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

### 6.1. Definitions

Unless specifically defined otherwise, all technical and scientific terms used herein have the same meaning as commonly understood by one of ordinary skill in the art to which this disclosure belongs. Unless mentioned otherwise, the techniques employed or contemplated herein are standard methodologies well known to one of ordinary skill in the art. The practice of the present disclosure will employ, unless otherwise indicated, conventional techniques of microbiology, tissue culture, molecular biology, chemistry, biochemistry and recombinant DNA technology, which are within the skill of the art. The materials, methods and examples are illustrative only and not limiting. The following is presented by way of illustration and is not intended to limit the scope of the disclosure.

In some embodiments, the numerical parameters set forth in the specification (into which the claims are incorporated in their entirety) are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

For convenience, certain terms employed in the entire application (including the specification, examples, and appended claims) are collected here. Unless defined otherwise, all technical and scientific terms used herein have the same meaning as commonly understood by one of ordinary skill in the art to which this disclosure belongs.

In some embodiments, numbers expressing quantities of ingredients, properties such as molecular weight, reaction conditions and results, and so forth, used to describe and claim certain embodiments of the present disclosure are to be understood as being modified in some instances by the term “about.” One of ordinary skill in the art would understand the meaning of the term “about” in the context of the value that it qualifies. In some embodiments, the term “about” is used to indicate that a value includes the standard deviation of the mean for the device or method being employed to determine the value. In some embodiments, the numerical parameters set forth in the specification (into which the claims are incorporated in their entirety) are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements.

As used herein, the term “administering” refers to the placement of a nanoparticle and/or a pharmaceutical composition comprising at least one nanoparticle into a mammalian tissue or a subject by a method or route that results in at least partial localization of the nanoparticle and/or composition at a desired site or tissue location. In some embodiments, nanoparticles comprising a lipid component and a modified RNA can be administered via an intradermal route, for example by injection. In some embodiments, at least a portion of the protein expressed by the modified RNA is localized to a desired target tissue or target cell location via intradermal administration. In some embodiments, nanoparticles comprising a lipid component and a modified RNA can be administered by topical administration or topical application. In some embodiments, nanoparticles comprising a lipid component and a modified RNA can be administered by topical administration or topical application on a wound. In some embodiments, at least a portion of the protein expressed by the modified RNA is localized to a desired target tissue or target cell location via topical administration. In some embodiments, protein expression resulting from the modified RNA administered via intradermal administration causes improved healing of a wound relative to healing in the absence of administration of the modified RNA. In some embodiments, protein expression resulting from the modified RNA administered via topical administration causes improved healing of a wound relative to healing in the absence of administration of the modified RNA.

The term “pharmaceutical composition” refers to a mixture that contains a therapeutically active component(s) and a carrier or excipient, such as a pharmaceutically acceptable carrier or excipient that is conventional in the art. For example, a pharmaceutical composition as used herein usually comprises at least a lipid component, a modified RNA according to the disclosure, and a suitable excipient.

The term “compound” includes all isotopes and isomers of the structure depicted. “Isotope” refers to atoms having the same atomic number but different mass numbers resulting from a different number of neutrons in the nuclei. For example, isotopes of hydrogen include tritium and deuterium. Further, a compound, salt, or complex of the present disclosure can be prepared in combination with solvent or water molecules to form solvates and hydrates by routine methods. “Isomer” means any geometric isomer, tautomer, zwitterion, stereoisomer, enantiomer, or diastereomer of a compound. Compounds may include one or more chiral centers and/or double bonds and may thus exist as stereoisomers, such as double-bond isomers or diastereomers. The present disclosure encompasses any and all isomers of the compounds described herein, including stereomerically pure forms and enantiomeric and stereoisomeric mixtures, e.g., racemates. Enantiomeric and stereomeric mixtures of compounds and means of resolving them into their component enantiomers or stereoisomers are well-known in the art.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition that “comprises,” “has” or “includes” one or more features is not limited to possessing only those one or more features and can cover other unlisted features. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element as essential to the practice of the present disclosure.

The term “consisting essentially of” allows for the presence of additional materials or steps that “do not materially affect the basic and novel characteristic(s)” of the claimed invention.

The term “consisting of” refers to compositions, methods, and respective components thereof as described herein, which are exclusive of any element not recited in that description of the embodiment.

The term “delivering” means providing an entity to a destination. For example, delivering a therapeutic to a subject may involve administering a pharmaceutical composition comprising at least one nanoparticle including the modified RNA to the subject (e.g., by an intradermal route or by a topical route). Administration of a pharmaceutical composition comprising at least one nanoparticle to mammalian tissue or a subject may involve contacting one or more cells with the pharmaceutical composition via intradermal administration (e.g., an intradermal injection). Administration of a pharmaceutical composition comprising at least one nanoparticle to mammalian tissue or a subject may involve contacting one or more cells with the pharmaceutical composition via topical administration or topical application.

The terms “disease” or “disorder” are used interchangeably herein, and refers to any alternation in state of the body or of some of the organs, interrupting or disturbing the performance of the functions and/or causing symptoms such as discomfort, dysfunction, distress, or even death to the person afflicted or those in contact with a person. A disease or disorder can also be related to a distemper, ailing, ailment, malady, sickness, illness, complaint, indisposition, or affection.

The term “effective amount” as used herein refers to the amount of therapeutic agent (for example, a modified RNA) or pharmaceutical composition sufficient to reduce at least one or more symptom(s) of the disease or disorder, or to provide the desired effect. For example, it can be the amount that induces a therapeutically significant reduction in a symptom or clinical marker associated with wound healing.

As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

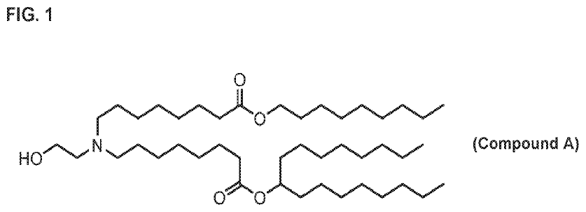

As used herein, the term “lipid component” is that component of a nanoparticle that includes one or more lipids. For example, the lipid component may include one or more cationic/ionizable, PEGylated, structural, or other lipids, such as phospholipids. In one embodiment, the lipid component comprises Compound A (FIG. 1). In one embodiment, the lipid component comprises dilinoleylmethyl-4-dimethylaminobutyrate (DLin-MC3-DMA).

As used herein, the term “modified RNA” refers to RNA molecules containing one, two, or more than two nucleoside modifications comparing to adenosine (A) ((2R,3R,4S,5R)-2-(6-amino-9H-purin-9-yl)-5-(hydroxymethyl)oxolane-3,4-diol), guanosine (G) (2-Amino-9-[3,4-dihydroxy-5-(hydroxymethyl)oxolan-2-yl]-3H-purin-6-one), cytidine (C) (4-amino-1-[3,4-dihydroxy-5-(hydroxymethyl)tetrahydrofuran-2-yl]pyrimidin-2-one), and uridine (U) (1-[(3R,4S,5R)-3,4-dihydroxy-5-(hydroxymethyl)oxolan-2-yl]pyrimidine-2,4-dione), or compared to AMP, GMP, CMP, and UMP, in RNA molecules, or a portion thereof. Non-limiting examples of nucleoside modifications are provided elsewhere in this specification. Where the nucleotide sequence of a particular claimed RNA is otherwise identical to the sequence of a naturally-existing RNA molecule, the modified RNA is understood to be an RNA molecule with at least one modification different from those existing in the natural counterpart. The difference can be either in the chemical change to the nucleoside/nucleotide or in the position of that change within the sequence. In one embodiment, the modified RNA is a modified messenger RNA (or “modified mRNA”). In some embodiments, a modified RNA includes at least one UMP that is modified to form N1-methyl-pseudo-UMP. In some embodiments, all UMPs in a modified RNA have been replaced by N1-methyl-pseudo-UMP.

As used herein, a “nanoparticle” is a particle comprising one or more lipids and one or more therapeutic agents. Nanoparticles are typically sized on the order of micrometers or smaller and may include a lipid bilayer. In some embodiments, the nanoparticle has a mean diameter (e.g., a hydrodynamic diameter) of between about 50 nm and about 100 nm, for example between about 60 nm and about 90 nm, between about 70 nm and about 90 nm, or between about 70 nm and about 85 nm in diameter, as measured by dynamic light scattering (see NIST Special Publication 1200-6, “Measuring the Size of Nanoparticles in Aqueous Media Using Batch Mode Dynamic Light Scattering”). In some embodiments, the nanoparticle has a mean hydrodynamic diameter of about 71 nm, 72 nm, 73 nm, 74 nm, 75 nm, 76 nm, 77 nm, 78 nm, 79 nm, 80 nm, 81 nm, 82 nm, 83 nm, 84 nm, 85 nm, 86 nm, 87 nm, 88 nm, 89 nm or 90 nm. In some embodiments, the therapeutic agent is a modified RNA. In some embodiments, the nanoparticles comprise Compound A as shown in FIG. 1 and a modified RNA. In some embodiments, the nanoparticles comprise dilinoleylmethyl-4-dimethylaminobutyrate (DLin-MC3-DMA) and a modified RNA.

As used herein, the “polydispersion index (pDI)” is the measure of the distribution of nanoparticle sizes in a nanoparticulate sample (see NIST Special Publication 1200-6, “Measuring the Size of Nanoparticles in Aqueous Media Using Batch Mode Dynamic Light Scattering”). In some embodiments, the polydispersity index is between about 0.01 and about 0.20, for example between about 0.03 and about 0.10, between about 0.04 and about 0.08, for example, about 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19 or 0.20.

As used herein, the “N:P ratio” is the molar ratio of ionizable (in the physiological pH range) nitrogen atoms in a lipid to phosphate groups in an RNA, e.g., in a nanoparticle including a lipid component and a modified RNA.

As used herein, the term “nucleic acid,” in its broadest sense, includes any compound and/or substance that comprises a polymer of nucleotides linked via a phosphodiester bond. These polymers are often referred to as oligonucleotides or polynucleotides, depending on the size. The terms “polynucleotide sequence” and “nucleotide sequence” are also used interchangeably herein.

As used herein, a “PEG lipid” or “PEGylated lipid” refers to a lipid comprising a polyethylene glycol component.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. Drug-approval agencies (e.g., EMA, US-FDA) provide guidance and approve pharmaceutically acceptable compounds, materials, compositions, and/or dosage forms. Examples are listed in Pharmacopeias.

The phrase “pharmaceutically acceptable excipient” is employed herein to refer to a pharmaceutically acceptable material chosen from a solvent, dispersion media, diluent, dispersion, suspension aid, surface active agent, isotonic agent, thickening or emulsifying agent, preservative, polymer, peptide, protein, cell, hyaluronidase, and mixtures thereof. In some embodiments, the solvent is an aqueous solvent.

As used herein, a “phospholipid” is a lipid that includes a phosphate moiety and one or more carbon chains, such as unsaturated fatty acid chains. A phospholipid may include one or more multiple (e.g., double or triple) bonds (e.g., one or more unsaturations). Particular phospholipids may facilitate fusion to a membrane. For example, a cationic phospholipid may interact with one or more negatively charged phospholipids of a membrane (e.g., a cellular or intracellular membrane). Fusion of a phospholipid to a membrane may allow one or more elements of a lipid-containing composition to pass through the membrane permitting, e.g., delivery of the one or more elements to a cell.

As used herein, “polypeptide” means a polymer of amino acid residues (natural or unnatural) linked together most often by peptide bonds. The term, as used herein, refers to proteins, polypeptides, and peptides of any size, structure, or function. A polypeptide may be a single molecule or may be a multi-molecular complex such as a dimer, trimer or tetramer. They may also comprise single chain or multichain polypeptides such as antibodies or insulin and may be associated or linked. Most commonly disulfide linkages are found in multichain polypeptides. The term polypeptide may also apply to amino acid polymers in which one or more amino acid residues are an artificial chemical analogue of a corresponding naturally occurring amino acid.

As used herein, “protein” is a polymer consisting essentially of any of the 20 amino acids. Although “polypeptide” is often used in reference to relatively large polypeptides, and “peptide” is often used in reference to small polypeptides, usage of these terms in the art overlaps and is varied. The terms “peptide(s)”, “protein(s)” and “polypeptide(s)” are sometime used interchangeably herein.

The term “subject” refers to an animal, for example a human, to whom treatment, including prophylactic treatment, with methods and compositions described herein, is provided. For treatment of those conditions or disease states which are specific for a specific animal such as a human subject, the term “subject” refers to that specific animal.

The term “tissue” refers to a group or layer of similarly specialized cells which together perform certain special functions.

As used herein, the terms “treat,” “treatment,” or “treating” refers to an amelioration or elimination of a disease or disorder, or at least one discernible symptom thereof. In some embodiments, “treatment” or “treating” refers to an amelioration or elimination of at least one measurable physical parameter, not necessarily discernible by the patient.

It should be understood that this disclosure is not limited to the particular methodology, protocols, and reagents, etc., described herein and as such can vary. The terminology used herein is for the purpose of describing particular embodiments only, and is not intended to limit the scope of the present disclosure, which is defined solely by the claims.

### 6.2. Lipid Components

In some embodiments, nanoparticles comprise a lipid component including Compound A (FIG. 1). In some embodiments, nanoparticles comprise a lipid component including dilinoleylmethyl-4-dimethylaminobutyrate (DLin-MC3-DMA). Additional compounds are disclosed in WO 2017/049245 A2 (see, e.g., compounds 1-147 in WO 2017/049245 A2), which is incorporated herein by reference in its entirety. The lipid components may also include a variety of other lipids such as a phospholipid, a structural lipid, and/or a PEG lipid.

Phospholipids

The lipid component of a nanoparticle may include one or more phospholipids, such as one or more (poly)unsaturated lipids. Phospholipids may assemble into one or more lipid bilayers. In general, phospholipids may include a phospholipid moiety and one or more fatty acid moieties.

Phospholipids useful in the compositions and methods may be selected from the non-limiting group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), and sphingomyelin. In some embodiments, a lipid component includes DSPC. In some embodiments, a lipid component includes DOPE. In some embodiments, a lipid component includes both DSPC and DOPE.

Structural Lipids

The lipid component of a nanoparticle may include one or more structural lipids. Structural lipids can be selected from, but are not limited to, cholesterol, fecosterol, sitosterol, ergosterol, campesterol, stigmasterol, brassicasterol, tomatidine, tomatine, ursolic acid, alpha-tocopherol, and mixtures thereof. In some embodiments, the structural lipid is cholesterol. In some embodiments, the structural lipid includes cholesterol and a corticosteroid (such as prednisolone, dexamethasone, prednisone, and hydrocortisone), or a combination thereof. In some embodiments, a lipid component includes cholesterol.

PEG Lipids

The lipid component of a nanoparticle may include one or more PEG or PEG-modified lipids. Such lipids may be alternately referred to as PEGylated lipids. A PEG lipid is a lipid modified with polyethylene glycol. A PEG lipid may be selected from the non-limiting group consisting of PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, DMG-PEG (1,2-dimyristoyl-rac-glycero-3-methoxypolyethylene glycol), obtainable from Avanti Polar Lipids, Alabaster, Ala.), DMG-PEG2000 (1,2-dimyristoyl-rac-glycero-3-methoxypolyethylene glycol-2000), PEG-DLPE, PEG-DMPE, PEG-DPPC, or a PEG-DSPE lipid. In some embodiments, a lipid component includes DMG-PEG. In some embodiments, a lipid component includes DMG-PEG2000.

### 6.3. Modified RNAs Encoding VEGF-A Polypeptides

It is of great interest in the fields of therapeutics, diagnostics, reagents and for biological assays to be able to deliver a nucleic acid, e.g., a ribonucleic acid (RNA) inside a cell, whether in vitro, in vivo, in situ, or ex vivo, such as to cause intracellular translation of the nucleic acid and production of an encoded polypeptide of interest.

Naturally occurring RNAs are synthesized from four basic ribonucleotides: ATP, CTP, UTP and GTP, but may contain post-transcriptionally modified nucleotides. Further, approximately one hundred different nucleoside modifications have been identified in RNA (Rozenski, J, Crain, P, and McCloskey, J., The RNA Modification Database: 1999 update, Nucl Acids Res, (1999) 27: 196-197).

According to the present disclosure, these RNAs are preferably modified as to avoid the deficiencies of other RNA molecules of the art (e.g., activating the innate immune response and rapid degradation upon administration). Hence, these polynucleotides are referred to as modified RNA. In some embodiments, the modified RNA avoids the innate immune response upon administration to a subject. In some embodiments, the half-life of the modified RNA is extended compared to an unmodified RNA.

In preferred embodiments, the RNA molecule is a messenger RNA (mRNA). As used herein, the term “messenger RNA” (mRNA) refers to any polynucleotide that encodes a polypeptide of interest and that is capable of being translated to produce the encoded polypeptide of interest in vitro, in vivo, in situ or ex vivo.

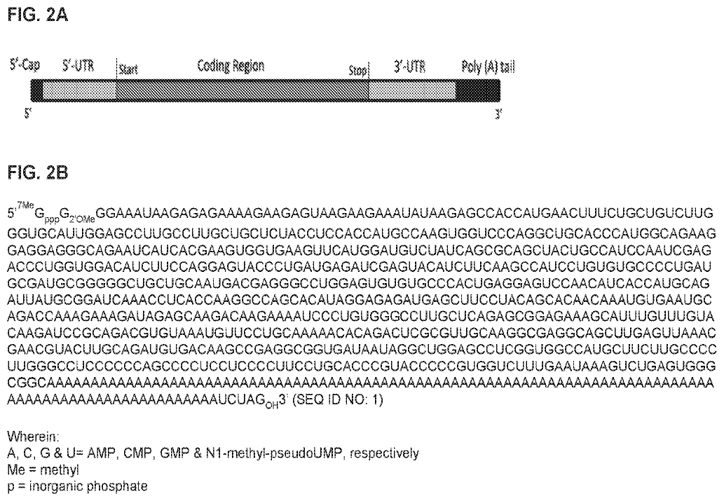

As depicted in FIG. 2A, traditionally, the basic components of an mRNA molecule include at least a coding region, a 5′ untranslated region (UTR), a 3′ untranslated region (UTR), a 5′ cap and a poly-(A) tail. Building on this wild-type modular structure, the present disclosure expands the scope of functionality of traditional mRNA molecules by providing polynucleotides or primary RNA constructs which maintain a modular organization, but which comprise one or more structural and/or chemical modifications or alterations that impart useful properties to the polynucleotide including, in some embodiments, the lack of a substantial induction of the innate immune response of a cell into which the polynucleotide is introduced.

The modified RNAs can include any useful modification relative to the standard RNA nucleotide chain, such as to the sugar, the nucleobase (e.g., one or more modifications of a nucleobase, such as by replacing or substituting an atom of a pyrimidine nucleobase with optionally substituted amino, optionally substituted thiol, optionally substituted alkyl (e.g., methyl or ethyl), or halo (e.g., chloro or fluoro)), or the internucleoside linkage (e.g., one or more modification to the phosphodiester backbone).

As non-limiting examples, in some embodiments, a modified RNA can include, for example, at least one uridine monophosphate (UMP) that is modified to form N1-methyl-pseudo-UMP. In some embodiments, the N1-methyl-pseudo-UMP is present instead of UMP in a percentage of the UMPs in the sequence of 0.1%, 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99.9%, and 100%. In some embodiments, all UMP have been replaced by N1-methyl-pseudo-UMP.

In some embodiments, modified RNAs comprise a modification to 5′ cap, such as a 5′ diguanosine cap. In some embodiments, modified RNAs comprise a modification to a coding region. In some embodiments, modified RNAs comprise a modification to a 5′ UTR. In some embodiments, modified RNAs comprise a modification to a 3′ UTR. In some embodiments, modified RNAs comprise a modification to a poly-(A) tail. In some embodiments, modified RNAs comprise any combination of modifications to a coding region, 5′ cap, 5′ UTR, 3′ UTR, or poly-(A) tail. In some embodiments, a modified RNA can optionally be treated with an alkaline phosphatase.

In some embodiments, a modified RNA encodes a Vascular Endothelial Growth Factor (VEGF) polypeptide, any one of a large family of VEGF proteins that play a central role in the regulation of wound healing in general. VEGF's roles also include activation of nitric oxide (NO) signaling, developmental and post-natal angiogenesis, tumor angiogenesis, arteriogenesis, endothelial replication, and as cell fate switch for multipotent cardiovascular progenitors.

It will be appreciated by those of skill in the art that for any particular VEGF gene there may exist one or more variants or isoforms. Non-limiting examples of VEGF-A polypeptides in accordance with the present disclosure are listed in Table 1. It will be appreciated by those of skill in the art that the sequences disclosed in Table 1 contain potential flanking regions. These are encoded in each nucleotide sequence either to the 5′ (upstream) or 3′ (downstream) of the open reading frame. The open reading frame is definitively and specifically disclosed by teaching the nucleotide reference sequence. It is also possible to further characterize the 5′ and 3′ flanking regions by utilizing one or more available databases or algorithms. Databases have annotated the features contained in the flanking regions of the NCBI sequences and these are available in the art.

TABLE 1
Homo sapiens VEGF-A mRNA isoforms.

| Description | NM Ref. | NP Ref. |
|---|---|---|
| Homo sapiens vascular endothelial | NM_001171623.1 | NP_001165094.1 |
| growth factor A (VEGF-A), transcript | | |
| variant 1, mRNA | | |
| Homo sapiens vascular endothelial | NM_001025366.2 | NP_001020537.2 |
| growth factor A (VEGF-A), transcript | | |
| variant 1, mRNA | | |
| Homo sapiens vascular endothelial | NM_001171624.1 | NP_001165095.1 |
| growth factor A (VEGF-A), transcript | | |
| variant 2, mRNA | | |
| Homo sapiens vascular endothelial | NM_003376.5 | NP_003367.4 |
| growth factor A (VEGF-A), transcript | | |
| variant 2, mRNA | | |
| Homo sapiens vascular endothelial | NM_001171625.1 | NP_001165096.1 |
| growth factor A (VEGF-A), transcript | | |
| variant 3, mRNA | | |
| Homo sapiens vascular endothelial | NM_001025367.2 | NP_001020538.2 |
| growth factor A (VEGF-A), transcript | | |
| variant 3, mRNA | | |
| Homo sapiens vascular endothelial | NM_001171626.1 | NP_001165097.1 |
| growth factor A (VEGF-A), transcript | | |
| variant 4, mRNA | | |
| Homo sapiens vascular endothelial | NM_001025368.2 | NP_001020539.2 |
| growth factor A (VEGF-A), transcript | | |
| variant 4, mRNA | | |
| Homo sapiens vascular endothelial | NM_001317010.1 | NP_001303939.1 |
| growth factor A (VEGF-A), transcript | | |
| variant 4, mRNA | | |
| Homo sapiens vascular endothelial | NM_001171627.1 | NP_001165098.1 |
| growth factor A (VEGF-A), transcript | | |
| variant 5, mRNA | | |
| Homo sapiens vascular endothelial | NM_001025369.2 | NP_001020540.2 |
| growth factor A (VEGF-A), transcript | | |
| variant 5, mRNA | | |
| Homo sapiens vascular endothelial | NM_001171628.1 | NP_001165099.1 |
| growth factor A (VEGF-A), transcript | | |
| variant 6, mRNA | | |
| Homo sapiens vascular endothelial | NM_001025370.2 | NP_001020541.2 |
| growth factor A (VEGF-A), transcript | | |
| variant 6, mRNA | | |
| Homo sapiens vascular endothelial | NM_001171629.1 | NP_001165100.1 |
| growth factor A (VEGF-A), transcript | | |
| variant 7, mRNA | | |
| Homo sapiens vascular endothelial | NM_001033756.2 | NP_001028928.1 |
| growth factor A (VEGF-A), transcript | | |
| variant 7, mRNA | | |
| Homo sapiens vascular endothelial | NM_001171630.1 | NP_001165101.1 |
| growth factor A (VEGF-A), transcript | | |
| variant 8, mRNA | | |
| Homo sapiens vascular endothelial | NM_001171622.1 | NP_001165093.1 |
| growth factor A (VEGF-A), transcript | | |
| variant 8, mRNA | | |
| Homo sapiens vascular endothelial | NM_001204385.1 | NP_001191314.1 |
| growth factor A (VEGF-A), transcript | | |
| variant 9, mRNA | | |
| Homo sapiens vascular endothelial | NM_001204384.1 | NP_001191313.1 |
| growth factor A (VEGF-A), transcript | | |
| variant 9, mRNA | | |
| Homo sapiens vascular endothelial | NM_001287044.1 | NP_001273973.1 |
| growth factor A (VEGF-A), transcript | | |
| variant 10, mRNA | | |

It will be appreciated by those of skill in the art that RNA molecules encoding VEGF-A polypeptides, e.g., a human VEGF-A polypeptide, can be designed according to the VEGF-A mRNA isoforms listed in Table 1. One of ordinary of skill in the art is generally familiar with the multiple isoforms of the remaining VEGF family members.

In one embodiment, the present disclosure provides for a modified RNA encoding a VEGF-A polypeptide (e.g., SEQ ID NO: 2). In some embodiments, a modified RNA encodes a VEGF-A polypeptide, wherein the modified RNA comprises any one of SEQ ID NOs: 1 and 3-5. In some embodiments, the modified RNA further comprises a 5′ cap, a 5′ UTR, a 3′ UTR, a poly(A) tail, or any combinations thereof. In some embodiments, the 5′ cap, the 5′ UTR, the 3′ UTR, the poly(A) tail, or any combinations thereof may include one or more modified nucleotides.

In some embodiments, a modified RNA encoding a VEGF-A polypeptide can have the structure as depicted in FIG. 2B, which is SEQ ID NO: 1. In some embodiments, a modified RNA encoding a VEGF-A polypeptide can have the sequence of any one of SEQ ID NOs: 3-5.

### 6.4. Compositions Comprising Lipid Component and Modified RNA

Some embodiments relate to nanoparticles that include a lipid component and a modified RNA.

In some embodiments, the lipid component of a nanoparticle may include Compound A (FIG. 1). In some embodiments, the lipid component of a nanoparticle may further include a phospholipid, a structural lipid, and/or a PEG lipid as disclosed herein. For example, in some embodiments, the lipid component of a nanoparticle may include DSPC, cholesterol, DMG-PEG, and mixtures thereof. The elements of the lipid component may be provided in specific fractions. In some embodiments, the lipid component of a nanoparticle includes Compound A, a phospholipid, a structural lipid, and a PEG lipid. In some embodiments, the lipid component of the nanoparticle includes from about 30 mol % to about 60 mol % Compound A, from about 0 mol % to about 30 mol % phospholipid, from about 18.5 mol % to about 48.5 mol % structural lipid, and from about 0 mol % to about 10 mol % of PEG lipid, provided that the total mol % does not exceed 100%. In some embodiments, the lipid component of the nanoparticle includes from about 35 mol % to about 55 mol % Compound A, from about 5 mol % to about 25 mol % phospholipid, from about 30 mol % to about 40 mol % structural lipid, and from about 0 mol % to about 10 mol % of PEG lipid. In some embodiment, the lipid component includes about 50 mol % Compound A, about 10 mol % phospholipid, about 38.5 mol % structural lipid, and about 1.5 mol % of PEG lipid. In some embodiments, the phospholipid may be DOPE. In some embodiments, the phospholipid may be DSPC. In some embodiments, the structural lipid may be cholesterol. In some embodiments, the PEG lipid may be DMG-PEG.

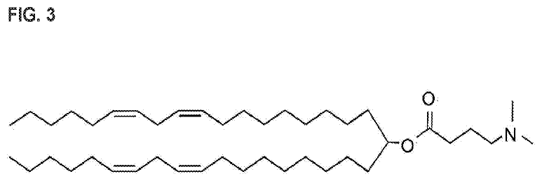

In some embodiments, the lipid component of a nanoparticle may include dilinoleylmethyl-4-dimethylaminobutyrate (DLin-MC3-DMA) (FIG. 3). In some embodiments, the lipid component of a nanoparticle may further include a phospholipid, a structural lipid, and/or a PEG lipid as disclosed herein. For example, in some embodiments, the lipid component of a nanoparticle may include DSPC, cholesterol, DMG-PEG (for example DMG-PEG2000), and mixtures thereof.

The elements of the lipid component may be provided in specific fractions. In some embodiments, the lipid component of a nanoparticle includes dilinoleylmethyl-4-dimethylaminobutyrate (DLin-MC3-DMA), a phospholipid, a structural lipid, and a PEG lipid. In some embodiments, the lipid component of the nanoparticle includes from about 30 mol % to about 60 mol % DLin-MC3-DMA, from about 0 mol % to about 30 mol % phospholipid, from about 18.5 mol % to about 48.5 mol % structural lipid, and from about 0 mol % to about 10 mol % of PEG lipid, provided that the total mol % does not exceed 100%. In some embodiments, the lipid component of the nanoparticle includes from about 35 mol % to about 55 mol % DLin-MC3-DMA, from about 5 mol % to about 25 mol % phospholipid, from about 30 mol % to about 40 mol % structural lipid, and from about 0 mol % to about 10 mol % of PEG lipid. In some embodiment, the lipid component includes about 50 mol % DLin-MC3-DMA, about 10 mol % phospholipid, about 38.5 mol % structural lipid, and about 1.5 mol % of PEG lipid. In some embodiments, the phospholipid may be DSPC. In some embodiments, the structural lipid may be cholesterol. In some embodiments, the PEG lipid may be DMG-PEG (for example DMG-PEG2000).

In some embodiments, the modified RNA component of a nanoparticle may include a modified RNA encoding a VEGF-A polypeptide as disclosed herein (e.g., SEQ ID NO: 2). In some embodiments, the modified RNA component of a nanoparticle may include the modified RNA comprising any one of SEQ ID NOs: 1 and 3-5. In some embodiments, the modified RNA component of a nanoparticle includes the modified RNA comprising SEQ ID NO: 3. In some embodiments, the modified RNA component of a nanoparticle includes the modified RNA comprising SEQ ID NO: 4. In some embodiments, the modified RNA further comprises a 5′ cap, a 5′ UTR, a 3′ UTR, a poly(A) tail, or any combinations thereof. In some embodiments, the 5′ cap, the 5′ UTR, the 3′ UTR, the poly(A) tail, or any combinations thereof may include one or more modified nucleotides.

In some embodiments, the relative amounts of the lipid component and the modified RNA in a nanoparticle may vary. In some embodiments, the wt/wt ratio of the lipid component to the modified RNA in a nanoparticle may be from about 5:1 to about 100:1, such as 5:1, 6:1, 7:1, 8:1, 9:1, 10:1, 11:1, 12:1, 13:1, 14:1, 15:1, 16:1, 17:1, 18:1, 19:1, 20:1, 25:1, 30:1, 35:1, 40:1, 45:1, 50:1, 60:1, 70:1, 80:1, 90:1, and 100:1. For example, the wt/wt ratio of the lipid component to the modified RNA may be from about 5:1 to about 40:1. In some embodiments, the wt/wt ratio is from about about 10:1 to about 20:1. In some embodiments, the wt/wt ratio is about 20:1. In some embodiments, the wt/wt ratio is about 10:1. In some embodiments, the wt/wt ratio is about 10.25:1.

In some embodiments, the relative amounts of the lipid component and the modified RNA in a nanoparticle may be provided by a specific N:P ratio. The N:P ratio of the composition refers to the molar ratio of nitrogen atoms in one or more lipids to the number of phosphate groups in an RNA. In general, a lower N:P ratio is preferred. In some embodiments, the N:P ratio may be from about 2:1 to about 30:1, such as 2:1, 3:1, 4:1, 5:1, 6:1, 7:1, 8:1, 9:1, 10:1, 12:1, 14:1, 16:1, 18:1, 20:1, 22:1, 24:1, 26:1, 28:1, or 30:1. In some embodiments, the N:P ratio may be from about 2:1 to about 8:1. For example, the N:P ratio may be about 3.0:1, about 3.5:1, about 4.0:1, about 4.5:1, about 5.0:1, about 5.5:1, about 5.67:1, about 6.0:1, about 6.5:1, or about 7.0:1. In some embodiments, the N:P ratio may be from about 2:1 to about 4:1. In some embodiments, the N:P ratio may be about 3:1.

Lipid nanoparticles can be prepared using methods well-known in the art (see, e.g., Belliveau et al., “Microfluidic synthesis of highly potent limit-size lipid nanoparticles for in vivo delivery of siRNA,” Mol. Ther. Nucleic Acids, 2012, 1(8):e37; Zhigaltsev et al., Bottom-up design and synthesis of limit size lipid nanoparticle systems with aqueous and triglyceride cores using millisecond microfluidic mixing,” Langmuir, 2012, 28(7):3633-3640).

In some embodiments, nanoparticles may additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Excipients can also include, without limitation, polymers, core-shell nanoparticles, peptides, proteins, cells, hyaluronidase, nanoparticle mimics and combinations thereof. Various excipients for formulating pharmaceutical compositions and techniques for preparing the composition are known in the art (see Remington: The Science and Practice of Pharmacy, 22^{nd} Edition, Edited by Allen, Loyd V., Jr, Pharmaceutical Press; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

In some embodiments, nanoparticles may comprise a pharmaceutically effective amount of a lipid component and a modified RNA, wherein the compositions further comprise a pharmaceutically acceptable excipient. In some embodiments, the pharmaceutically acceptable excipient is chosen from a solvent, dispersion media, diluent, dispersion, suspension aid, surface active agent, isotonic agent, thickening or emulsifying agent, preservative, core-shell nanoparticles, polymer, peptide, protein, cell, hyaluronidase, and mixtures thereof. In some embodiments, the solvent is an aqueous solvent. In some embodiments, the solvent is a non-aqueous solvent.

The present disclosure also provides for a pharmaceutical composition comprises one or more lipid nanoparticles comprising a lipid component and a modified RNA as disclosed herein, and a pharmaceutically acceptable excipient. In some embodiments, pharmaceutical compositions comprise a plurality of lipid nanoparticles as disclosed herein and a pharmaceutically acceptable excipient. In some embodiments, the pharmaceutically acceptable excipient is chosen from a solvent, dispersion media, diluent, dispersion, suspension aid, surface active agent, isotonic agent, thickening or emulsifying agent, preservative, core-shell nanoparticles, polymer, peptide, protein, cell, hyaluronidase, and mixtures thereof. In some embodiments, the solvent is an aqueous solvent. In some embodiments, the solvent is a non-aqueous solvent.

### 6.5. Improving Wound Healing in a Subject

VEGF-A pathways play a central role in wound healing processes, including revascularization of damaged tissues, improving vascular permeability, and formation of new blood vessels. It is an aim of the present disclosure to treat subjects who suffers from diseases resulting from defective wound healing processes.

In some embodiments, nanoparticles according to this disclosure are administered to a subject who suffers from a disease that affects vascular structures. Vascular structures are most commonly injured by penetrating trauma, burns, or surgery. Diabetes impairs numerous components of wound healing, and a patient with diabetic wound healing generally has altered blood flow due to vascular dysfunction. Accordingly, a subject with skin ulcer including diabetic ulcers usually has decreased or delayed wound healing. In some embodiments, nanoparticles as disclosed herein are administered to a subject who suffers from diabetes. In the context of this disclosure, a wound can be, for example, a surgical wound, a burn, an abrasive wound, a skin biopsy site, a chronic wound, an injury (e.g., a traumatic injury wound), a graft wound, a diabetic wound, a diabetic ulcer (e.g., diabetic foot ulcer), a pressure ulcer, bed sore, and combinations thereof.

In some embodiments, nanoparticles comprising a lipid component and a modified RNA (e.g., SEQ ID NO: 1, SEQ ID NO: 3, SEQ ID NO: 4, or SEQ ID NO: 5) may be used to improve wound healing in a mammalian tissue or a subject.

In some embodiments, nanoparticles as disclosed herein may be used to induce neovascularization in a mammalian tissue ora subject. In some embodiments, nanoparticles as disclosed herein may be used to induce angiogenesis in a mammalian tissue or a subject.

Yet in some embodiments, nanoparticles as disclosed herein may be used to treat a vascular injury from trauma or surgery. In some embodiments, nanoparticles as disclosed herein may be used to treat a disease involving skin grafting and tissue grafting.

Other aspects of the disclosure relate to administration of the nanoparticles to subjects in need thereof. In some embodiments, nanoparticles as disclosed herein are administered via an intradermal route to improve wound healing of a mammalian tissue or a subject.

In certain embodiments, nanoparticles as disclosed herein may be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of modified RNA per subject body weight per day, one or more times a day, to obtain the desired therapeutic effect.

In some embodiments, nanoparticles as disclosed herein are administered to a subject in a single administration. In some embodiments, nanoparticles as disclosed herein are administered to the subject, at a fixed-dosage in multiple (e.g., two, three, four, five, six, seven, eight, nine, ten, eleven, twelve, thirteen, fourteen, fifteen, sixteen, seventeen, eighteen, nineteen, twenty, or more) administrations. In each of the embodiments in this paragraph, the “multiple administrations” can be separated from each other by short (1-5 mins), medium (6-30 minutes), or long (more than 30 minutes, hours, or even days) intervals of time.

The nanoparticles may be administered to a subject using any dosage of administration effective for treating a disease, disorder, and/or condition. The exact dosage required will vary from subject to subject, depending on the species, age, and general condition of the subject, the severity of the disease, the particular formulation, its mode of administration, its mode of activity, and the like. It will be understood, however, that the total daily usage of the compositions may be decided by the attending physician within the scope of sound medical judgment. The specific pharmaceutically effective dose level for any particular patient will depend upon a variety of factors including the severity of the disease, the specific composition employed, the age, body weight, general health, sex and diet of the patient, the time of administration, route of administration (e.g. intradermal or topical), the duration of the treatment, and like factors well-known in the medical arts.

All of the claims in the claim listing are herein incorporated by reference into the specification in their entireties as additional embodiments.

### 7. EXAMPLES

### Example 1

Preparation of Nanoparticle and Citrate Saline Compositions

Compound A Lipid Nanoparticles (Compound A-LNPs): Stock solution of lipids in ethanol were prepared from Compound A, distearoyl phosphatidylcholine (DSPC, Avanti Polar Lipids), cholesterol (Sigma), and 1,2-dimyristoyl-rac-glycero-3-methoxypolyethylene glycol-2000 (DMG-PEG2000 from NOF Corporation). The lipids were mixed in ethanol 99.5% to a total lipid concentration of 12.5 mM. The composition was Compound A, DSPC, Cholesterol, DMG-PEG2000 at the ratio of 50:10:38.5:1.5% mol. The VEGF-A modified RNA (e.g., SEQ ID NO: 1, SEQ ID NO: 3, SEQ ID NO: 4, or SEQ ID NO: 5) was thawed and diluted to 6.25 mM in sodium acetate buffer and HyClone water at a concentration corresponding to a total lipid:mRNA weight ratio of 11:1 (charge ratio nitrogen:phosphate (N:P) of 3) in the final formulation. The final formulation after dilution was as follows:

TABLE 2
Compound A-LNP 1:11 (N:P = 3),
mRNA concentration 0.06 mg/mL

| | Amount |
|---|---|
| LNP Component | (mg/mL) |
| Modified VEGF-A RNA | 0.06 |
| Compound A | 0.37 |
| DSPC | 0.08 |
| Cholesterol | 0.16 |
| DMG-PEG2000 | 0.04 |

The Compound A-LNP compositions were prepared by rapidly mixing ethanol solution containing the lipids and aqueous solution of a modified VEGF-A RNA on a microfluidic device, followed by dialysis in phosphate buffered saline (PBS). Briefly, the modified VEGF-A RNA solution and the lipid solution were injected into a microfluidic mixing device (NanoAssemblr™ (Precision Nanosystems)) at a volumetric ratio of aqueous to ethanol 3:1 and flow rates of 12-14 mL/min using two syringes, which were controlled by syringe pumps. Ethanol was removed by dialyzing Compound A-LNP compositions against PBS buffer overnight using membranes with 10 KD cutoff. Compound A-LNP compositions were characterized by particle size (63 nm), polydispersity index (0.10) and encapsulation (96%). Compound A-LNP compositions were diluted to a final concentration of 0.06 mg/mL with PBS and filtered sterile. Compound A-LNP compositions were stored refrigerated. The size and polydispersity of Compound A-LNPs was determined by dynamic light scattering using a Zetasizer Nano ZS (Malvern Instruments Ltd) and the encapsulation and concentration of mRNA in the Compound A-LNP formulations were determined using the RiboGreen assay.

DLin-MC3-DMA Lipid Nanoparticles (MC3-LNPs): Stock solution of lipids in ethanol were prepared from DLin-MC3-DMA (synthesized as described in Jayaraman, M., et al., Angew Chem Int Ed Engl, 2012, 51(34), p. 8529-33), distearoyl phosphatidylcholine (DSPC, Avanti Polar Lipids), cholesterol (Sigma), and 1,2-dimyristoyl-rac-glycero-3-methoxpolyethylene glycol-2000 (DMG-PEG2000 from NOF Corporation). The lipids were mixed in ethanol 99.5% to a total lipid concentration of 12.5 mM. The composition was DLin-MC3-DMA, DSPC, Cholesterol, DMG-PEG2000 at the ratio of 50:10:38.5:1.5% mol. The VEGF-A modified RNA (e.g., SEQ ID NO: 1, SEQ ID NO: 3, SEQ ID NO: 4, or SEQ ID NO: 5) was thawed and diluted to 6.25 mM in sodium acetate buffer (pH 5) and HyClone water at a concentration corresponding to a total lipid:mRNA weight ratio of 10.25:1 (charge ratio nitrogen:phosphate (N:P) of 3) in the final formulation. The final formulations after dilution were as follows:

TABLE 3
MC3-LNP 1:10.25 (N:P = 3),
mRNA concentration 0.075 mg/mL

| | Amount |
|---|---|
| LNP Component | (mg/mL) |
| VEGF-A modified RNA | 0.075 |
| DLin-MC3-DMA | 0.42 |
| DSPC | 0.10 |
| Cholesterol | 0.20 |
| DMG-PEG2000 | 0.05 |

MC3-Lipid nanoparticle (LNP) compositions: The LNP compositions were prepared by rapidly mixing ethanol solution containing the lipids and aqueous solution of VEGF-A modified RNA on a microfluidic device, followed by dialysis in phosphate buffered saline (PBS). Briefly, the VEGF-A modified RNA solution and the lipid solution were injected into a microfluidic mixing device (NanoAssemblr™ (Precision Nanosystems)) at a volumetric ratio of aqueous to ethanol 3:1 and flow rates of 12-14 mL/min using two syringes, which were controlled by syringe pumps. Following microfluidic mixing, the MC3-LNPs were dialyzed overnight against phosphate buffered saline (pH 7.4) using Slide-A-Lyzer™ G2 dialysis cassettes with a molecular weight cut-off of 10k (Thermo Scientific).

MC3-LNP compositions were characterized by particle size (77 to 85 nm), VEGF-A modified RNA concentration (0.076 to 0.1 mg/mL), polydispersity index (0.04 to 0.08) and encapsulation (96 to 98%). MC3-LNP compositions were diluted to a final concentration of 0.075 mg/mL with PBS and filtered sterile. MC3-LNP compositions were stored refrigerated. The size and polydispersity of MC3-LNPs was determined by dynamic light scattering using a Zetasizer Nano ZS (Malvern Instruments Ltd) and the encapsulation and concentration of mRNA in the MC3-LNP formulations were determined using the RiboGreen assay.

Citrate saline compositions: Citrate saline compositions were prepared by diluting a thawed modified VEGF-A RNA solution with HyClone water and a concentrated buffer solution to a final composition of 10 mM sodium citrate and 130 mM sodium chloride at pH 6.5.

### Example 2

Assessment of Wound Healing Following Intradermal Injection of Human VEGF-A Modified RNA in Mouse

A MC3 lipid nanoparticle composition comprising a modified VEGF-A RNA and DLin-MC3-DMA was prepared as in Example 1 with VEGF-A modified RNA having the sequence of SEQ ID NO: 4.

A second MC3 lipid nanoparticle composition comprising a non-translatable (NT) modified VEGF-A RNA and DLin-MC3-DMA was prepared as in Example 1 with VEGF-A modified RNA having the sequence of SEQ ID NO: 6.

Male db/db mice were used. These mice are an established model of Type II diabetes and have impaired wound healing as compared to wild-type mice.

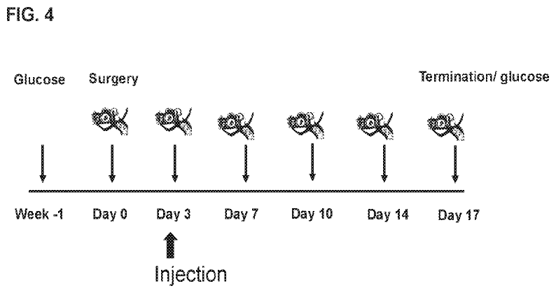

FIG. 4 provides a timeline of the surgical procedure, treatment, and observation time points of the study. Glucose and body weight were measured the week before the start of study and at termination. The mice were randomized according to fasting (4 hours) glucose levels, which were measured the week before surgery. The mice were anesthetized with isoflurane before undergoing surgery. The surgical procedure was started by removing the hair on the back of the mice by using clippers and hair removal cream. One wound on the back of each mouse was made by creating a mark with a 10 mm biopsy punch and then cutting it out. The wound was covered by a tegaderm transparent dressing to protect the wound. A self-adhering elastic bandage was placed around the mouse covering the wound area, and an injection of analgesic (Tamgesic at 0.08 mg/ml) was administered at a dosage of 0.05-0.1 mg/kg according to the weight of the mouse.

The mice were separated into three treatment groups: (a) citrate/saline solution (10 mM sodium citrate and 130 mM sodium chloride at pH 6.5) (n=7), (b) mRNA VEGF NT 3 μg MC3 (non-translatable VEGF-A modified RNA formulated with MC3 LNP) (n=7), and (c) mRNA VEGF 3 μg MC3 (modified VEGF-A RNA formulated with MC3 LNP) (n=7). The treatment solutions were injected intradermally as 4 injections (10 μl each) around the wound (40 μl total), as a single dose at day 3 (FIG. 4).

The wounds were examined every 3^{rd} or 4^{th} day until all wounds were healed, for up to 17 days. The tegaderm was removed and replaced after examination. Pictures of the wounds were taken with a Canon camera at a fixed distance from the wound. The wound area was determined by tracing the wound margin using the image analyzing software Image J, and then calculated as a percent area of the baseline area. Statistical evaluation was done with an unpaired, two-sided t-test, and p-values<0.05 were considered significant.

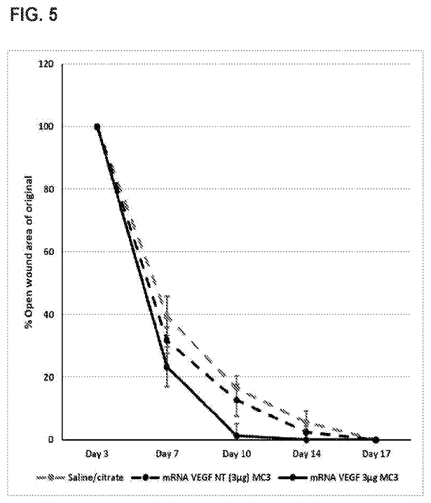

As shown in the results in Table 4 and FIG. 5, intradermal injection of a lipid nanoparticle composition comprising 3 μg of modified VEGF-A RNA formulated with MC3 significantly improved wound healing when compared to a lipid nanoparticle composition comprising 3 μg of non-translatable VEGF-A formulated with MC3, or citrate saline, as demonstrated by the decrease in the percent of open wound area.

TABLE4
% of open wound original area from baseline (Day 3)

| | Day | Day | Day | Day | Day |
|---|---|---|---|---|---|
| | 3 | 7 | 10 | 14 | 17 |
| Formulation | (%) | (%) | (%) | (%) | (%) |
| Saline/citrate | 100.0 | 39.6 | 16.4 | 5.6 | 0.0 |
| mRNA VEGF-A NT | 100.0 | 31.7 | 12.7 | 2.4 | 0.0 |
| (3 μg) MC3 | | | | | |
| mRNA VEGF-A | 100.0 | 23.3 | 1.3 | 0.0 | 0.0 |
| (3 μg) MC3 | | | | | |

### Example 3

Assessment of Wound Healing Following Topical Application of Human VEGF-A Modified RNA in Mouse

A MC3 lipid nanoparticle composition comprising a modified VEGF-A RNA and DLin-MC3-DMA was prepared as in Example 1 with VEGF-A modified RNA having the sequence of SEQ ID NO: 4.

Male db/db mice were used. These mice are an established model of Type II diabetes and have impaired wound healing as compared to wild-type mice.

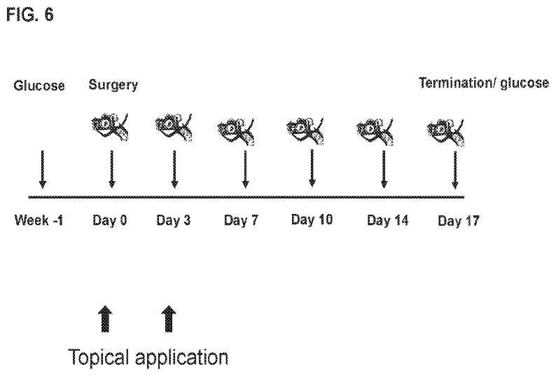

FIG. 6 provides a timeline of the surgical procedure, treatment, and observation time points of the study. Glucose and body weight were measured the week before the start of study and at termination. The mice were randomized according to fasting (4 hours) glucose levels, which were measured the week before surgery. The mice were anesthetized with isoflurane before undergoing surgery. The surgical procedure was started by removing the hair on the back of the mice by using clippers and hair removal cream. One wound on the back of each mouse was made by creating a mark with a 10 mm biopsy punch and then cutting it out. The wound was covered by a tegaderm transparent dressing to protect the wound. A self-adhering elastic bandage was placed around the mouse covering the wound area, and an injection of analgesic (Tamgesic at 0.08 mg/ml) was administered at a dosage of 0.05-0.1 mg/kg according to the weight of the mouse.

The mice were separated into two treatment groups: (a) citrate/saline solution (10 mM sodium citrate and 130 mM sodium chloride at pH 6.5) (n=5), and (b) mRNA VEGF 3 μg MC3 (n=5). The treatment solutions were administered via topical application through a needle inserted through the tegaderm at day 0 and day 3 (FIG. 6).

The wounds were examined every 3^{rd} or 4^{th} day until all wounds were healed, for up to 17 days. The tegaderm was removed and replaced after examination. Pictures of the wounds were taken with a Canon camera at a fixed distance from the wound. The wound area was determined by tracing the wound margin using the image analyzing software Image J, and then calculated as a percent area of the baseline area. Statistical evaluation was done with an unpaired, two-sided t-test, and p-values<0.05 were considered significant.

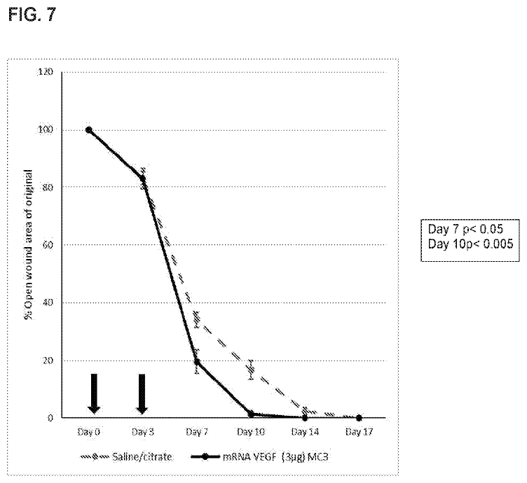

As shown in the results in Table 5 and FIG. 7, topical application of a lipid nanoparticle composition comprising 3 μg of modified VEGF-A RNA formulated with MC3 significantly improved wound healing when compared to applied saline citrate, as demonstrated by the decrease in the percent of open wound area.

TABLE 5
% of open wound original area from baseline (Day 0)

| | Day | Day | Day | Day | Day | Day |
|---|---|---|---|---|---|---|
| | 0 | 3 | 7 | 10 | 14 | 17 |
| Formulation | (%) | (%) | (%) | (%) | (%) | (%) |
| Saline/citrate | 100.0 | 83.3 | 34.1 | 16.7 | 2.3 | 0.0 |
| mRNA VEGF-A | 100.0 | 83.0 | 19.6 | 1.3 | 0.0 | 0.0 |
| (3 μg) MC3 | | | | | | |

### Example 4

Quantification of Human VEGF-A (hVEGF-A) Protein in Pig Skin

Citrate saline compositions and nanoparticle compositions comprising a VEGF-A modified RNA and either Compound A or DLin-MC3-DMA (MC3) were prepared as in Example 1. The citrate saline composition, Compound A nanoparticle composition, and MC3 nanoparticle composition were all prepared with VEGF-A modified RNA having the sequence of SEQ ID NO: 4.

Wound preparation: The stratum corneum of the skin of Gottingen mini pigs was removed with a scalpel blade, and the rest of the epidermis was removed by using a Cotech mini grinder.

Topical administration of a nanoparticle composition comprising a modified VEGF-A RNA and MC3: 40 μl containing 3 μg of mRNA in the MC3 nanoparticle formulation was applied on three areas where the epidermis was removed from the skin of the pigs. The tissue was removed 5-6 hours after application, snap frozen in liquid nitrogen, and stored at −80° C. until the time the analysis was performed. The procedure was repeated on four pigs.

Intradermal infection of a nanoparticle composition comprising a modified VEGF-A RNA and MC3: 40 μl containing 3 μg of mRNA in the MC3 nanoparticle formulation was administered by intradermal injection (using an insulin syringe) into three sites where the epidermis was removed from the skin of the pigs. The tissue was removed 5-6 hours after application, snap frozen in liquid nitrogen, and stored at −80° C. until the time the analysis was performed. The procedure was repeated on four pigs.

Topical administration of a nanoparticle composition comprising a VEGF-A RNA and Compound A: 50 μl containing 3 μg of mRNA in the Compound A nanoparticle formulation was applied on three areas where the epidermis was removed from the skin of the pigs. The tissue was removed 5 hours after application, snap frozen in liquid nitrogen, and stored at −80° C. until the time the analysis was performed. The procedure was repeated on five pigs.

Topical administration of citrate/saline (control): 50 μl containing 100 μg mRNA in the citrate/saline formulation was applied on three areas where the epidermis was removed from the skin of the pigs. The tissue was removed 5 hours after application, snap frozen in liquid nitrogen, and stored at −80° C. until the time analysis was performed. The procedure was repeated on three pigs.

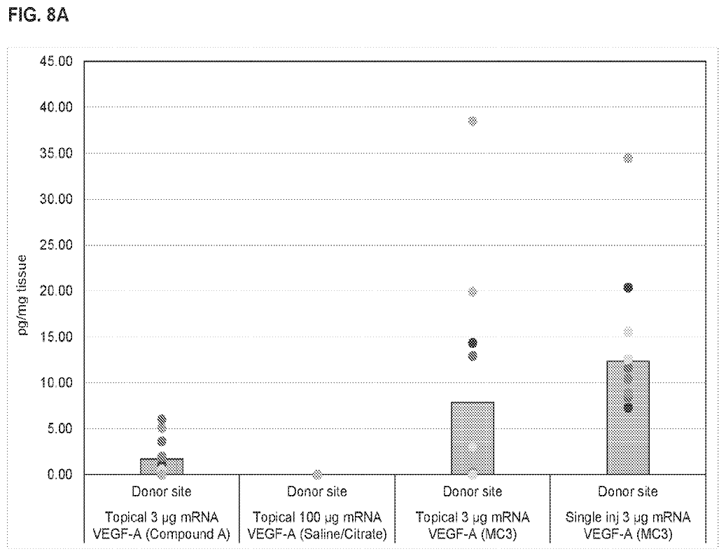

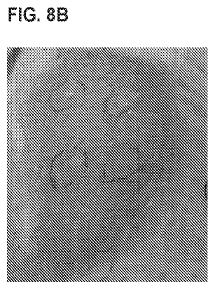

Each tissue sample was analyzed for expression of human VEGF-A protein using ELISA, and the results summarized in Table 6 (FIG. 8A-B).

TABLE 6

| | Topical 3 μg mRNA | | Topical 100 μg | | Topical 3 μg mRNA | | Single inj 3 μg mRNA | |
|---|---|---|---|---|---|---|---|---|
| | VEGF-A formulated | | mRNA VEGF-A | | VEGF-A formulated | | VEGF-A formulated | |
| | with Compound A | | (Saline/Citrate) | | with MC3 | | with MC3 | |
| | | pg of | | pg of | | pg of | | pg of |
| | | VEGF- | | VEGF- | | VEGF- | | VEGF- |
| | | A/mg | | A/mg | | A/mg | | A/mg |
| | | skin | | skin | | skin | | skin |
| | pig # | tissue | pig # | tissue | pig # | tissue | pig # | tissue |
| | Pig 1 | 3.64 | Pig 1 | 0.00 | Pig 1 | 0.00 | Pig 1 | 11.62 |
| | Pig 1 | 0.69 | Pig 1 | 0.00 | Pig 1 | 5.22 | Pig 1 | 8.49 |
| | Pig 2 | 5.08 | Pig 1 | 0.00 | Pig 1 | 0.30 | Pig 1 | 5.15 |
| | Pig 2 | 0.00 | Pig 2 | 0.00 | Pig 2 | 14.36 | Pig 2 | 20.35 |
| | Pig 2 | 0.00 | Pig 2 | 0.00 | Pig 2 | 3.04 | Pig 2 | 15.56 |
| | Pig 3 | 0.96 | Pig 2 | 0.00 | Pig 2 | 38.49 | Pig 2 | 34.49 |
| | Pig 3 | 1.95 | Pig 3 | 0.00 | Pig 3 | 0.16 | Pig 3 | 10.44 |
| | Pig 3 | 6.02 | Pig 3 | 0.00 | Pig 3 | 12.91 | Pig 3 | 8.43 |
| | Pig 4 | 0.56 | Pig 3 | 0.00 | Pig 3 | 19.92 | Pig 3 | 5.12 |
| | Pig 4 | 1.69 | | | Pig 4 | 0.00 | Pig 4 | 8.90 |
| | Pig 4 | 1.09 | | | Pig 4 | 0.00 | Pig 4 | 7.30 |
| | Pig 5 | 0.57 | | | Pig 4 | 0.00 | Pig 4 | 12.59 |
| | Pig 5 | 0.00 | | | | | | |
| | Pig 5 | 1.96 | | | | | | |
| Mean | | 1.73 | | 0.00 | | 7.87 | | 12.37 |
| SEM | | 0.51 | | 0.00 | | 3.42 | | 2.37 |
| # of | | 5 | | 3 | | 4 | | 4 |
| animals | | | | | | | | |

FIG. 8A shows the production of human VEGF-A protein (hVEGF-A) in pig tissue 5-6 hours after topical application and single injection treatments with the modified VEGF-A RNA formulated in an MC3 LNP, and the production of hVEGF-A protein in pig tissue 5 hours after topical application treatment with the modified VEGF-A RNA formulated with a Compound A LNP. Treatment with the citrate saline composition did not result in the production of hVEGF-A protein. FIG. 8B depicts a wound on pig skin, with the drawn circles indicating the sites of topical treatment with modified VEGF-A RNA formulated in MC3.

8. SEQUENCES
8.1. SEQ ID NO: 1: A modified RNA encoding VEGF-A
(SEQ ID NO: 1)
5′^{7Me}GₚₚₚG_{2′OMe}GGAAAUAAGAGAGAAAAGAAGAGUAAGAAGAAAUAUAAG
AGCCACCAUGAACUUUCUGCUGUCUUGGGUGCAUUGGAGCCUUGCCU
UGCUGCUCUACCUCCACCAUGCCAAGUGGUCCCAGGCUGCACCCAUG
GCAGAAGGAGGAGGGCAGAAUCAUCACGAAGUGGUGAAGUUCAUGGA
UGUCUAUCAGCGCAGCUACUGCCAUCCAAUCGAGACCCUGGUGGACA
UCUUCCAGGAGUACCCUGAUGAGAUCGAGUACAUCUUCAAGCCAUCCU
GUGUGCCCCUGAUGCGAUGCGGGGGCUGCUGCAAUGACGAGGGCCU
GGAGUGUGUGCCCACUGAGGAGUCCAACAUCACCAUGCAGAUUAUGC
GGAUCAAACCUCACCAAGGCCAGCACAUAGGAGAGAUGAGCUUCCUAC
AGCACAACAAAUGUGAAUGCAGACCAAAGAAAGAUAGAGCAAGACAAGA
AAAUCCCUGUGGGCCUUGCUCAGAGCGGAGAAAGCAUUUGUUUGUAC
AAGAUCCGCAGACGUGUAAAUGUUCCUGCAAAAACACAGACUCGCGUU
GCAAGGCGAGGCAGCUUGAGUUAAACGAACGUACUUGCAGAUGUGAC
AAGCCGAGGCGGUGAUAAUAGGCUGGAGCCUCGGUGGCCAUGCUUCU
UGCCCCUUGGGCCUCCCCCCAGCCCCUCCUCCCCUUCCUGCACCCGU
ACCCCCGUGGUCUUUGAAUAAAGUCUGAGUGGGCGGCAAAAAAAAAAA
AAAAAAAAAAAAAAAAAAAAAAAAAAAAAAAAAAAAAAAAAAAAAAAAAAA
AAAAAAAAAAAAAAAAAAAAAAAAAAAAAAAAAAAAAAUCUAG_{OH}3′
Wherein:
A, C, G & U = AMP, CMP, GMP & N1-methyl-pseudoUMP,
respectively
Me = methyl
p = inorganic phosphate
8.2. SEQ ID NO: 2: Amino acid sequence of human
VEGF-A isoform VEGF-165
(SEQ ID NO: 2)
MNFLLSWVHWSLALLLYLHHAKWSQAAPMAEGGGQNHHEVVKFMDVYQR
SYCHPIETLVDIFQEYPDEIEYIFKPSCVPLMRCGGCCNDEGLECVPTE
ESNITMQIMRIKPHQGQHIGEMSFLQHNKCECRPKKDRARQENPCGPCS
ERRKHLFVQDPQTCKCSCKNTDSRCKARQLELNERTCRCDKPRR
8.3. SEQ ID NO: 3: A modified RNA encoding VEGF-A
(SEQ ID NO: 3)
5′^{7Me}GₚₚₚG_{2′OMe}AGGAAAUAAGAGAGAAAAGAAGAGUAAGAAGAAAUAUAA
GAGCCACCAUGAACUUUCUGCUGUCUUGGGUGCAUUGGAGCCUUGCC
UUGCUGCUCUACCUCCACCAUGCCAAGUGGUCCCAGGCUGCACCCAU
GGCAGAAGGAGGAGGGCAGAAUCAUCACGAAGUGGUGAAGUUCAUGG
AUGUCUAUCAGCGCAGCUACUGCCAUCCAAUCGAGACCCUGGUGGAC
AUCUUCCAGGAGUACCCUGAUGAGAUCGAGUACAUCUUCAAGCCAUCC
UGUGUGCCCCUGAUGCGAUGCGGGGGCUGCUGCAAUGACGAGGGCC
UGGAGUGUGUGCCCACUGAGGAGUCCAACAUCACCAUGCAGAUUAUG
CGGAUCAAACCUCACCAAGGCCAGCACAUAGGAGAGAUGAGCUUCCUA
CAGCACAACAAAUGUGAAUGCAGACCAAAGAAAGAUAGAGCAAGACAA
GAAAAUCCCUGUGGGCCUUGCUCAGAGCGGAGAAAGCAUUUGUUUGU
ACAAGAUCCGCAGACGUGUAAAUGUUCCUGCAAAAACACAGACUCGCG
UUGCAAGGCGAGGCAGCUUGAGUUAAACGAACGUACUUGCAGAUGUG
ACAAGCCGAGGCGGUGAUAAUAGGCUGGAGCCUCGGUGGCCAUGCUU
CUUGCCCCUUGGGCCUCCCCCCAGCCCCUCCUCCCCUUCCUGCACCC
GUACCCCCGUGGUCUUUGAAUAAAGUCUGAGUGGGCGGCAAAAAAAAA
AAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAUCUAG_{OH}3′
Wherein:
A, C, G & U = AMP, CMP, GMP & N1-methyl-pseudoUMP,
respectively
Me = methyl
p = inorganic phosphate
8.4. SEQ ID NO: 4: A modified RNA encoding VEGF-A
(VEGF-01-012)
(SEQ ID NO: 4)
5′^{7Me}GₚₚₚGGGAAAUAAGAGAGAAAAGAAGAGUAAGAAGAAAUAUAAGAGC
CACCAUGAACUUUCUCCUUUCUUGGGUGCAUUGGAGCCUUGCCUUGU
UACUCUACCUCCACCACGCCAAGUGGUCCCAGGCCGCACCCAUGGCA
GAAGGAGGAGGGCAGAAUCAUCACGAAGUGGUGAAGUUCAUGGACGU
CUAUCAGCGCAGCUACUGCCAUCCAAUCGAGACACUGGUGGACAUCUU
CCAGGAGUACCCUGAUGAGAUCGAGUACAUCUUCAAGCCAUCCUGUG
UGCCCCUGAUGCGAUGCGGCGGCUGCUGCAAUGACGAGGGCCUGGA
GUGUGUGCCUACUGAGGAGUCCAACAUCACCAUGCAGAUUAUGCGGA
UCAAACCUCACCAAGGCCAGCACAUAGGAGAGAUGAGCUUCCUACAGC
ACAACAAAUGUGAAUGCAGACCAAAGAAAGAUAGAGCAAGACAAGAGAA
UCCCUGUGGGCCUUGCUCAGAGCGGAGAAAGCAUUUGUUUGUACAAG
AUCCGCAGACGUGUAAAUGUUCCUGCAAGAACACAGACUCGCGUUGCA
AGGCGAGGCAGCUUGAGUUAAACGAACGUACUUGCAGAUGUGACAAG
CCGAGGCGGUGAUAAUAGGCUGGAGCCUCGGUGGCCAUGCUUCUUGC
CCCUUGGGCCUCCCCCCAGCCCCUCCUCCCCUUCCUGCACCCGUACC
CCCGUGGUCUUUGAAUAAAGUCUGAGUGGGCGGCAAAAAAAAAAAAAA
AAAAAAAAAAAAAAAAAAAAAAAAAAAAAAAAAAAAAAAAAAAAAAAAAA
AAAAAAAAAAAAAAAAAAAAAAAAAAAAAAAAAAAAUCUAG3′
Wherein:
A, C, G & U = AMP, CMP, GMP & N1-methyl-pseudoUMP,
respectively
p = inorganic phosphate
8.5. SEQ ID NO: 5: A modified RNA encoding VEGF-A
(SEQ ID NO: 5)
5′^{7Me}GₚₚₚAGGAAAUAAGAGAGAAAAGAAGAGUAAGAAGAAAUAUAAGAGC
CACCAUGAACUUUCUGCUGUCUUGGGUGCAUUGGAGCCUUGCCUUGC
UGCUCUACCUCCACCAUGCCAAGUGGUCCCAGGCUGCACCCAUGGCA
GAAGGAGGAGGGCAGAAUCAUCACGAAGUGGUGAAGUUCAUGGAUGU
CUAUCAGCGCAGCUACUGCCAUCCAAUCGAGACCCUGGUGGACAUCU
UCCAGGAGUACCCUGAUGAGAUCGAGUACAUCUUCAAGCCAUCCUGU
GUGCCCCUGAUGCGAUGCGGGGGCUGCUGCAAUGACGAGGGCCUGG
AGUGUGUGCCCACUGAGGAGUCCAACAUCACCAUGCAGAUUAUGCGG
AUCAAACCUCACCAAGGCCAGCACAUAGGAGAGAUGAGCUUCCUACAG
CACAACAAAUGUGAAUGCAGACCAAAGAAAGAUAGAGCAAGACAAGAAA
AUCCCUGUGGGCCUUGCUCAGAGCGGAGAAAGCAUUUGUUUGUACAA
GAUCCGCAGACGUGUAAAUGUUCCUGCAAAAACACAGACUCGCGUUGC
AAGGCGAGGCAGCUUGAGUUAAACGAACGUACUUGCAGAUGUGACAA
GCCGAGGCGGUGAUAAUAGGCUGGAGCCUCGGUGGCCAUGCUUCUUG
CCCCUUGGGCCUCCCCCCAGCCCCUCCUCCCCUUCCUGCACCCGUAC
CCCCGUGGUCUUUGAAUAAAGUCUGAGUGGGCGGCAAAAAAAAAAAAA
AAAAAAAAAAAAAAAAAAAAAAAAAAAAAAAAAAAAAAAAAAAAAAAAAA
AAAAAAAAAAAAAAAAAAAAAAAAAAAAAAAAAAAAAUCUAG3′
Wherein:
A, C, G & U = AMP, CMP, GMP & N1-methyl-pseudoUMP,
respectively
p = inorganic phosphate
8.6. SEQ ID NO: 6: A non-translatable VEGF-A
modified RNA
(SEQ ID NO: 6)
5′^{7Me}GₚₚₚGGGAAAUAAGAGAGAAAAGAAGAGUAAGAAGAAAUAUAAGAG
CCACCACGAACUUUGUGCUCUCUUGGGUGCAUUGGAGCCUUGCCUUGC
UGCUCUACCUCCACCACGCCAAGUGGUCCCAGGCCGCACCCACGGCA
GAAGGAGGAGGGCAGAAUCAUCACGAAGUGGUGAAGUUCACGGACGU
CUAUCAGCGCAGCUACUGCCAUCCAAUCGAGACCCUCGUGGACAUCUU
CCAGGAGUACCCUCACGAGAUCGAGUACAUCUUCAAGCCAUCCUGUGU
GCCCCUGACGCGACGCGGGGGCUGCUGCAACGACGAGGGCCUCGAG
UGUGUGCCCACCGAGGAGUCCAACACCACCACGCAGAUUACGCGGAU
CAAACCUCACCAAGGCCAGCACAUAGGAGAGACGAGCUUCCUACAGCA
CAACAAACGUGAACGCAGACCAAAGAAAGAUAGAGCAAGACAAGAAAAU
CCCUGUGGGCCUUGCUCAGAGCGGAGAAAGCAUUUGUUUGUACAAGA
UCCGCAGACGUGUAAACGUUCCUGCAAAAACACAGACUCGCGUUGCAA
GGCGAGGCAGCUUGAGUUAAACGAACGUACUUGCAGACGUGACAAGC
CGAGGCGGUGAUAAUAGGUUGGAGCCUCGGUGGCCACGCUUCUUGCC
CCUUGGGCCUCCCCCCAGCCCCUCCUCCCCUUCCUGCACCCGUACCC
CCGUGGUCUUUGAAUAAAGUCUGAGUGGGCGGCAAAAAAAAAAAAAAA
AAAAAAAAAAAAAAAAAAAAAAAAAAAAAAAAAAAAAAAAAAAAAAAAAA
AAAAAAAAAAAAAAAAAAAAAAAAAAAAAAAAAAA3′
Wherein:
A, C, G & U = AMP, CMP, GMP & N1-methyl-pseudoUMP,
respectively
p = inorganic phosphate

1. A nanoparticle comprising
(i) a lipid component comprising dilinoleylmethyl-4-dimethylaminobutyrate (DLin-MC3-DMA), and
(ii) a modified RNA comprising any one of SEQ ID NOs: 1 and 3-5, encoding a VEGF-A polypeptide of SEQ ID NO: 2.

2. The nanoparticle according to claim 1, wherein the lipid component further comprises a phospholipid, a structural lipid, and/or a PEG lipid.

3. (canceled)

4. The nanoparticle according to claim 2, wherein the phospholipid is selected from the group consisting of 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof;
the structural lipid is selected from the group consisting of cholesterol, fecosterol, sitosterol, ergosterol, campesterol, stigmasterol, brassicasterol, tomatidine, ursolic acid, alpha-tocopherol, and mixtures thereof; and/or
the PEG lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, DMG-PEG (1,2-dimyristoyl-rac-glycero-3-methoxypolyethylene glycol), DMG-PEG2000 (1,2-dimyristoyl-rac-glycero-3-methoxypolyethylene glycol-2000), and mixtures thereof.

5. The nanoparticle according to claim 1, wherein the lipid component further comprises a phospholipid that is DSPC, a structural lipid that is cholesterol, and/or a PEG lipid that is DMG-PEG.

6. The nanoparticle according to claim 1, wherein the ratio of ionizable nitrogen atoms in the lipid to the number of phosphate groups in the RNA (N:P ratio) is from 2:1 to 30:1.

7. (canceled)

8. The nanoparticle according to claim 1, wherein the wt/wt ratio of the lipid component to the modified RNA is from 5:1 to 100:1.

9.-12. (canceled)

13. A pharmaceutical composition comprising
(a) at least one nanoparticle comprising (i) a lipid component comprising dilinoleylmethyl-4-dimethylaminobutyrate (DLin-MC3-DMA), and (ii) a modified RNA comprising any one of SEQ ID NOs: 1 and 3-5, encoding a VEGF-A polypeptide of SEQ ID NO: 2; and
(b) a pharmaceutically acceptable excipient.

14.-24. (canceled)

25. The pharmaceutical composition according to claim 13, wherein the pharmaceutically acceptable excipient is chosen from a solvent, dispersion media, diluent, dispersion, suspension aid, surface active agent, isotonic agent, thickening or emulsifying agent, preservative, polymer, peptide, protein, cell, hyaluronidase, and mixtures thereof.

26. A method for promoting and/or improving wound healing, comprising administering to a subject in need thereof an effective amount of the nanoparticle according to claim 1.

27. The method according to claim 26, wherein the administration results in production of a VEGF-A polypeptide of SEQ ID NO: 2 in plasma or tissue of the subject.

28. The method according to claim 27, wherein the VEGF-A polypeptide is detected in the plasma and/or tissue within 5 or 6 hours after administration of the nanoparticle or pharmaceutical composition to the subject.

29. The method according to claim 27, wherein the administration results in production of more than 1 μg/mg of the VEGF-A polypeptide in the subject.

30. The method according to claim 26, wherein the nanoparticle or the pharmaceutical composition is administered intradermally.

31. The method according to claim 26, wherein the nanoparticle or the pharmaceutical composition is administered topically to a wound.

32. The method according to claim 26, wherein the nanoparticle is administered at a dosage level sufficient to deliver from 0.01 mg/kg to 10 mg/kg of modified RNA per subject body weight.

33. The method according to claim 26, wherein the administration increases production of a VEGF-A polypeptide of SEQ ID NO: 2 by a factor of 1 to 100, as compared to administration of the modified RNA in a citrate saline buffer to the subject.

34. The method according to claim 26, wherein the subject suffers from diabetes.

35. The method according to claim 26, wherein the wound is a surgical wound, a burn, an abrasive wound, a skin biopsy site, a chronic wound, an injury, a graft wound, a diabetic wound, a diabetic ulcer, a pressure ulcer, bed sore, or combinations thereof.

36. A method for inducing neovascularization comprising administering to a subject in need thereof an effective amount of the nanoparticle according to claim 1.

37. A method for inducing angiogenesis comprising administering to a subject in need thereof an effective amount of the nanoparticle according to claim 1.

38. A method for increasing capillary and/or arteriole density comprising administering to a subject in need thereof an effective amount of the nanoparticle according to claim 1.

39. A method for promoting and/or improving wound healing, comprising topically administering to a wound in a subject in need thereof an effective amount of a nanoparticle or pharmaceutical composition thereof comprising
(i) a lipid component, and
(ii) a modified RNA comprising any one of SEQ ID NOs: 1 and 3-5, encoding a VEGF-A polypeptide of SEQ ID NO: 2.

40. The method according to claim 39, wherein the lipid component comprises a compound having the structure

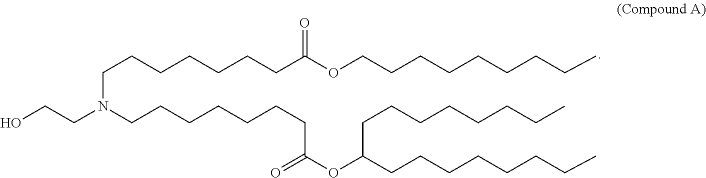 [SMILES: CCCCCCCCCOC(=O)CCCCCCCN(CCO)CCCCCCCC(=O)OC(CCCCCCCC)CCCCCCCC]

41. The method according to claim 39, wherein the lipid component comprises dilinoleylmethyl-4-dimethylaminobutyrate.

42. (canceled)

43. A method for promoting and/or improving wound healing, comprising topically administering to a wound in a subject in need thereof an effective amount of a nanoparticle or a pharmaceutical composition thereof comprising
(i) a lipid component comprising dilinoleylmethyl-4-dimethylaminobutyrate, and
(ii) modified RNA comprising any one of SEQ ID NOs: 1 and 3-5, encoding a VEGF-A polypeptide of SEQ ID NO: 2.

44.-52. (canceled)


SEQUENCE LISTING (6 sequences)

SEQ ID NO: 1 - RNA, 845 nt, Artificial Sequence
  source: /note=“Description of Artificial Sequence Synthetic polynucleotide”
  modified_base (1)..(1): 5′ 7MeGppp, wherein Me = methyl and p = inorganic phosphate
  source: /note=“Within this sequence A = AMP, C = CMP, G = GMP, U = N1-methyl-pseudoUMP”
  modified_base (1)..(845): Monophosphate or N1-methyl-pseudo monophosphate nucleotide
  modified_base (2)..(2): G2′OMe, wherein Me is methyl
  modified_base (845)..(845): GOH 3′
  source: /note=“See specification as filed for detailed description of substitutions and preferred embodiments”
ggggaaauaagagagaaaagaagaguaagaagaaauauaagagccaccaugaacuuucugcugucuugggugcauuggagccuugccuugcugcucuaccuccaccaugccaaguggucccaggcugcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaccaaagaaagauagagcaagacaagaaaaucccugugggccuugcucagagcggagaaagcauuuguuuguacaagauccgcagacguguaaauguuccugcaaaaacacagacucgcguugcaaggcgaggcagcuugaguuaaacgaacguacuugcagaugugacaagccgaggcggugauaauaggcuggagccucgguggccaugcuucuugccccuugggccuccccccagccccuccuccccuuccugcacccguacccccguggucuuugaauaaagucugagugggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaucuag

SEQ ID NO: 2 - protein, 191 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 3 - RNA, 846 nt, Artificial Sequence
  source: /note=“Description of Artificial Sequence Synthetic polynucleotide”
  modified_base (1)..(1): 5′ 7MeGppp, wherein Me = methyl and p = inorganic phosphate
  source: /note=“Within this sequence A = AMP, C = CMP, G = GMP, U = N1-methyl-pseudoUMP”
  modified_base (1)..(846): Monophosphate or N1-methyl-pseudo monophosphate nucleotide
  modified_base (2)..(2): G2′OMe, wherein Me is methyl
  modified_base (846)..(846): GOH 3′
  source: /note=“See specification as filed for detailed description of substitutions and preferred embodiments”
ggaggaaauaagagagaaaagaagaguaagaagaaauauaagagccaccaugaacuuucugcugucuugggugcauuggagccuugccuugcugcucuaccuccaccaugccaaguggucccaggcugcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaccaaagaaagauagagcaagacaagaaaaucccugugggccuugcucagagcggagaaagcauuuguuuguacaagauccgcagacguguaaauguuccugcaaaaacacagacucgcguugcaaggcgaggcagcuugaguuaaacgaacguacuugcagaugugacaagccgaggcggugauaauaggcuggagccucgguggccaugcuucuugccccuugggccuccccccagccccuccuccccuuccugcacccguacccccguggucuuugaauaaagucugagugggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaucuag

SEQ ID NO: 4 - RNA, 845 nt, Artificial Sequence
  source: /note=“Description of Artificial Sequence Synthetic polynucleotide”
  modified_base (1)..(1): 5′ 7MeGppp, wherein Me = methyl and p = inorganic phosphate
  source: /note=“Within this sequence A = AMP, C = CMP, G = GMP, U = N1-methyl-pseudoUMP”
  modified_base (1)..(845): Monophosphate or N1-methyl-pseudo monophosphate nucleotide
  source: /note=“See specification as filed for detailed description of substitutions and preferred embodiments”
ggggaaauaagagagaaaagaagaguaagaagaaauauaagagccaccaugaacuuucuccuuucuugggugcauuggagccuugccuuguuacucuaccuccaccacgccaaguggucccaggccgcacccauggcagaaggaggagggcagaaucaucacgaaguggugaaguucauggacgucuaucagcgcagcuacugccauccaaucgagacacugguggacaucuuccaggaguacccugaugagaucgaguacaucuucaagccauccugugugccccugaugcgaugcggcggcugcugcaaugacgagggccuggagugugugccuacugaggaguccaacaucaccaugcagauuaugcggaucaaaccucaccaaggccagcacauaggagagaugagcuuccuacagcacaacaaaugugaaugcagaccaaagaaagauagagcaagacaagagaaucccugugggccuugcucagagcggagaaagcauuuguuuguacaagauccgcagacguguaaauguuccugcaagaacacagacucgcguugcaaggcgaggcagcuugaguuaaacgaacguacuugcagaugugacaagccgaggcggugauaauaggcuggagccucgguggccaugcuucuugccccuugggccuccccccagccccuccuccccuuccugcacccguacccccguggucuuugaauaaagucugagugggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaucuag

SEQ ID NO: 5 - RNA, 845 nt, Artificial Sequence
  source: /note=“Description of Artificial Sequence Synthetic polynucleotide”
  modified_base (1)..(1): 5′ 7MeGppp, wherein Me = methyl and p = inorganic phosphate
  source: /note=“Within this sequence A = AMP, C = CMP, G = GMP, U = N1-methyl-pseudoUMP”
  modified_base (1)..(845): Monophosphate or N1-methyl-pseudo monophosphate nucleotide
  source: /note=“See specification as filed for detailed description of substitutions and preferred embodiments”
gaggaaauaagagagaaaagaagaguaagaagaaauauaagagccaccaugaacuuucugcugucuugggugcauuggagccuugccuugcugcucuaccuccaccaugccaaguggucccaggcugcacccauggcagaaggaggagggcagaaucaucacgaaguggugaaguucauggaugucuaucagcgcagcuacugccauccaaucgagacccugguggacaucuuccaggaguacccugaugagaucgaguacaucuucaagccauccugugugccccugaugcgaugcgggggcugcugcaaugacgagggccuggagugugugcccacugaggaguccaacaucaccaugcagauuaugcggaucaaaccucaccaaggccagcacauaggagagaugagcuuccuacagcacaacaaaugugaaugcagaccaaagaaagauagagcaagacaagaaaaucccugugggccuugcucagagcggagaaagcauuuguuuguacaagauccgcagacguguaaauguuccugcaaaaacacagacucgcguugcaaggcgaggcagcuugaguuaaacgaacguacuugcagaugugacaagccgaggcggugauaauaggcuggagccucgguggccaugcuucuugccccuugggccuccccccagccccuccuccccuuccugcacccguacccccguggucuuugaauaaagucugagugggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaucuag

SEQ ID NO: 6 - RNA, 840 nt, Artificial Sequence
  source: /note=“Description of Artificial Sequence Synthetic polynucleotide”
  modified_base (1)..(1): 5′ 7MeGppp, wherein Me = methyl and p = inorganic phosphate
  source: /note=“Within this sequence A = AMP, C = CMP, G = GMP, U = N1-methyl-pseudoUMP”
  modified_base (1)..(840): Monophosphate or N1-methyl-pseudo monophosphate nucleotide
  source: /note=“See specification as filed for detailed description of substitutions and preferred embodiments”
ggggaaauaagagagaaaagaagaguaagaagaaauauaagagccaccacgaacuuugugcucucuugggugcauuggagccuugccuugcugcucuaccuccaccacgccaaguggucccaggccgcacccacggcagaaggaggagggcagaaucaucacgaaguggugaaguucacggacgucuaucagcgcagcuacugccauccaaucgagacccucguggacaucuuccaggaguacccucacgagaucgaguacaucuucaagccauccugugugccccugacgcgacgcgggggcugcugcaacgacgagggccucgagugugugcccaccgaggaguccaacaccaccacgcagauuacgcggaucaaaccucaccaaggccagcacauaggagagacgagcuuccuacagcacaacaaacgugaacgcagaccaaagaaagauagagcaagacaagaaaaucccugugggccuugcucagagcggagaaagcauuuguuuguacaagauccgcagacguguaaacguuccugcaaaaacacagacucgcguugcaaggcgaggcagcuugaguuaaacgaacguacuugcagacgugacaagccgaggcggugauaauagguuggagccucgguggccacgcuucuugccccuugggccuccccccagccccuccuccccuuccugcacccguacccccguggucuuugaauaaagucugagugggcggcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa